(19) United States
(12) Patent Application Publication
(10) Pub. No.: US 2024/0382076 A1
(43) Pub. Date: Nov. 21, 2024

(54) FOLD-DOWN MECHANISM FOR AN ENDOSCOPE

(71) Applicant: Konstantin Bob, Weinheim (DE)

(72) Inventors: Konstantin Bob, Weinheim (DE); Andreas Gründl, Starnberg (DE)

(73) Assignee: Konstantin Bob, Weinheim (DE)

(21) Appl. No.: 18/570,427

(22) PCT Filed: Jun. 15, 2022
(86) PCT No.: PCT/EP2022/066401

§ 371 Date: Dec. 14, 2023

(30) Foreign Application Priority Data
Jun. 15, 2021 (DE) 10 2021 115 475.8

(51) Int. Cl. A61B 1/005

(52) U.S. Cl. CPC A61B 1/0056; A61B 1/0057

(57) ABSTRACT

An endoscope with a fold-down mechanism for holding an endoscope head at the distal end of an endoscope section so that it can be folded down or bent, with a number of segments which can be actively angularly adjusted relative to one another by way of at least one actuating element, which, in the side view, have two end faces which are aligned with respect to one another and a cylindrical jacket which defines a wedge back section at a cylindrical jacket section with maximum axial length, and which form at least one working channel in the longitudinal direction of the endoscope. The working channel in a wedge back section of each segment breaks through the cylinder jacket in the radial direction of the endoscope, and the segments are connected in the wedge back section by a flexible connecting plate in the longitudinal direction of the endoscope.

### CROSS-REFERENCE TO RELATED APPLICATIONS

This application is a U.S. National Phase application of PCT International Application No. PCT/EP2022/066401, filed Jun. 15, 2022, which claims the benefit of German Patent Application No. 10 2021 115 475.8, filed Jun. 15, 2021, both of which are incorporated by reference herein for all purposes.

### FIELD OF THE INVENTION

The present disclosure relates to a fold-down mechanism and in particular an endoscope with such a fold-down mechanism for holding an endoscope head at the distal end (facing the body of a patient) of the endoscope so that it can be folded down or bent, with a number of axially successive and essentially cylindrical (or elliptical) segments which can be actively angularly adjusted relative to one another by means of at least one actuating element, which, in the side view, have two end faces which are aligned essentially wedge-shaped with respect to one another and a cylindrical jacket which defines a wedge back section at a cylindrical jacket section with maximum axial length, and which form at least one working channel in the longitudinal direction of the endoscope for the insertion/passing through of a surgical instrument.

### BACKGROUND OF THE INVENTION

Endoscopes are medical tools for the visual exploration of cavities in a patient's body. They basically have optical devices on the distal end, i.e. user-averted or patient-body-facing end of the endoscope (also referred to as endoscope head), and optionally a working channel, which extends from a proximal (user-facing) endoscope section or extracorporeal endoscope section or extracorporeal endoscope handle through a flexible or rigid (in particular rigid) endoscope shaft (connected thereto) to the endoscope head and enables the extracorporeal insertion and use of a medical instrument such as forceps, scissors, needle, snare, knife and the like.

Such endoscopes can optionally be provided with additional capabilities, for example, by placing a cap or sleeve on the distal end of the endoscope/endoscope head radially on the outside of the endoscope head, which is provided or equipped with specific functions/functional elements, whereby the endoscope can be used not only for exploration and/or as an access for therapeutic applications, but also itself as a minimally invasive instrument for performing a surgical procedure. Alternatively, however, it is also envisaged that special endoscopes for very specific medical applications can be integrally equipped with such capabilities, such special designs, however, only being suited for this particular application.

Various diagnostic and/or therapeutic procedures require imaging, for example, imaging and/or, if necessary, therapeutic techniques on the patient's bile and/or pancreatic duct and hepatic ducts. As Vater's papilla, which forms the common exit of the bile duct and pancreatic duct into the duodenum, protrudes laterally into the duodenum, conventional prograde (looking in the longitudinal direction of the endoscope) endoscopes are unsuitable for the approach and insertion of surgical instruments into the bile duct, as there is not enough swivel space in the narrow duodenum (3 to 4 cm of diameter) to align their prograde optics and the working channel in a sideways-looking position, as the typical bending radius of such radius of such devices is around 6 cm. In conventional prograde endoscopes, this bending radius or pivoting is often achieved by so-called “deflectors” (sections of the endoscope shaft that can be actively bent proximally in front of the endoscope head).

### PRIOR ART

From the prior art (e.g. US 2010/228086 A), specially manufactured duodenoscopes are known for this purpose, which have a lateral (looking sideways) or retrograde (looking backwards) optics (also referred to as “side optics”) as well as a laterally directed or opening working channel. Alternatively, at a prograde (opening in the axial direction) exit of the working channel of such duodenoscopes, a so-called Albarran lever can be provided which enables targeted guidance/deflection of an instrument guided in the working channel by manual pivoting. The sideways arrangement of the functional units, in particular the optics and lighting, on the endoscope head enables imaging and treatment in the duodenum area while making optimal use of the available space.

However, such endoscopes with side optics are very complex and expensive to manufacture and have therefore been developed and manufactured as reusable devices up to now. The curved working channel of such endoscopes as well as the complex and undercut design of the Albarran lever have also proven to be difficult to sterilize in practice and the sterilization process has proven to be too material-tiring for the sensitive devices, so that only disinfection is possible after a procedure with such a duodenoscope. As a result, a bacterial lawn (biofilm) can remain in the working channel and/or parallel auxiliary channels for water, energy, etc. of the endoscope after a procedure. If this biofilm then dissolves during a subsequent procedure, for example, because an instrument is pushed through the working channel, it can enter the bile duct and/or pancreatic duct of the subsequent patient and cause serious inflammation and even sepsis in the patient.

Another disadvantage of such devices is that they can only be used for a few very specific procedures in the area of the duodenum, as neither the optics nor the working channel can be directed in a prograde direction. In addition, navigation in the body with sideways looking endoscopes is generally rather difficult, as looking ahead always requires bending the deflector immediately in front of the endoscope head by approximately 90°, which in turn requires more space in the transverse direction of the endoscope, which is only available in the stomach.

Furthermore, from DE 10 2018 110 620 A1 a fold-down mechanism for holding an endoscope head so that it can be folded down or bent, is known with a number of axially successive segments which can be actively, i.e. manually controlled, angularly adjusted relative to one another by means of an actuating element and which define a working channel in the axial direction. The segments are designed as elliptical or cylindrical sections with wedge-shaped end faces aligned with each other, whereby two directly adjacent segments are aligned with each other in such a way that they are axially supported or rest on each other at their respective cylinder jacket sections of maximum axial length, thereby creating a hinge or joint contact at the support or resting point. This design makes it possible to pivot the distal endoscope head into a radial alignment within a very narrow radius in such a way that the distal end face of the endoscope head does not or only slightly radially protrudes the outer circumference of the endoscope shaft. It should be noted that the fold-down mechanism constructed in this way can also be regarded functionally as a component of the endoscope head and thus divides the endoscope head into a distal and a proximal head section, whereby the proximal head section (proximal last segment) is firmly connected to the endoscope shaft (possibly via an additional deflector with an active bending radius different to that of the fold-down mechanism) and the distal head section (distal last segment) can be actively (manually controlled) pivoted/folded down towards the proximal head section.

The disadvantage of this mechanism, however, is that instruments inserted into the working channel may hit the segments, get caught and thus blocked, particularly due to the very narrow folding/deflection radius, and therefore often have to be moved back and forth several times in order to be inserted through the working channel. This makes it difficult to use the endoscope.

### SUMMARY OF THE INVENTION

The task underlying the disclosure is to avoid or reduce disadvantages of the prior art. In particular, a fold-down mechanism for an endoscope is to be provided, which enables an endoscope optic in the endoscope head to be aligned both forwards and, if very little space is required, laterally or possibly even backwards and which also ensures easy use in conjunction with medical instruments.

The problem on which the disclosure is based is solved by a fold-down mechanism and, in particular, by a (medical) endoscope that includes the features of the independent claims.

Fold-down mechanisms are based on a common inventive idea, as explained subsequently in more detail. A fold-down mechanism is provided which has a number of, at least one, preferably several, wedge-like segments (fold-down mechanism vertebral bodies) arranged longitudinally to one another with respect to the endoscope, which together form a working channel extending through the fold-down mechanism and which is/are connected to the distal end face of the endoscope shaft and/or to one another on one side in such hinge-like manner, that the number of (in particular several) segments can be bent relative to the distal end face and/or to each other via a corresponding joint in order to fold down/bend the fold-down mechanism (comparable to the finger of a human hand). If the fold-down mechanism is folded down/bent, the joints between the respective segments and/or to the distal end face of the endoscope shaft are located on an outer radius of curvature of the fold-down mechanism and thus on a side (circumferential section of the endoscope) on which an instrument advanced through the working channel slides off/is deflected against a wall of the working channel. Since the instrument can get caught and blocked at edges or gaps in the wall of the working channel, which are present at contact points between adjacent segments, or at least cannot be pushed in evenly (with uniform force/speed), a slide-off plate/slide-off band device is provided on the side (circumferential section) on which the instrument slides off, especially in the case of a bend greater than 0° (i.e. in the area of the joints), which is arranged on the side (circumferential section) of the outer radius of curvature of the working channel (forming a boundary/a wall section of the working channel formed by the segments) and replaces/forms or covers the joints and furthermore minimizes or covers edges and/or gaps (facing the working channel) between the respective adjacent segments on the outer radius of curvature of the fold-down mechanism in order to provide a continuous, smooth working channel sliding surface (in the longitudinal direction of the endoscope) for the instrument along the fold-down mechanism.

At this point, it should be noted that endoscopes of the present type (including the present endoscope according to the present disclosure) can optionally be equipped with actively actuable/bendable distal shaft sections, so-called deflectors, which are usually arranged between the endoscope head (i.e. proximal to the fold-down mechanism) and the usually passively bendable (flexible) endoscope shaft section. The main difference between the two mechanisms, which can be actively actuated separately from each other, is the desired, function-dependent bending radius. While the deflector basically serves to better follow the hollow organ or the course of the organ cavity (e.g. bowel, esophagus/trachea, etc.) when inserting the endoscope into a patient's hollow organ and, if necessary, approach a hollow organ wall for treatment purposes, the fold-down mechanism is configured to enable the endoscope head or a distal portion thereof to be folded down within the hollow organ quasi perpendicular to the hollow organ wall without the hollow organ having to be unduly widened.

Compared to deflecting, the latter requires a (significantly) smaller bending/deflection radius, with design consequences such as larger gaps between the segments on the outside of the deflection, which may then require additional design measures to eliminate them. Such measures are not necessary with deflectors and are also not stimulated by deflectors. In other words, an endoscope according to the disclosure may have the fold-down mechanism with a first maximum bending/deflection radius and may have a deflector provided proximal thereto with a second maximum bending/deflection radius, which is larger than the first maximum bending/deflection radius.

Optionally (in particular with regard to the linking of the segments to each other and the gaps occurring therebetween), the endoscope head or head portion may be considered as a (not necessarily wedge-shaped) segment and/or a distal end of the endoscope section proximally upstream of the fold-down mechanism can be considered as the most proximally arranged end (not necessarily wedge-shaped) segment. In this case, the fold-down mechanism may have a single wedge-like segment, with a plurality of wedge-like segments preferably being provided.

In the following, the solution to the problem is first described in more detail.

More precisely, the problem is solved by a fold-down mechanism for holding an endoscope head at the distal end/end section (facing away from the user/facing towards the patient's body) of an endoscope (shaft section), so that it can be folded down or bent, with a number of (a single or several) axially successive, substantially cylindrical (including oval/elliptical) segments which can be actively (manually controlled) angularly adjusted/folded down relative to one another by means of at least one actuating element and each of which has two substantially wedge-shaped end faces aligned with respect to one another and a cylindrical jacket which defines a wedge back section/wedge back region at a section of the cylinder jacket with maximum axial length, and which form at least one (inner) working channel in the longitudinal direction of the endoscope, the working channel in the wedge back section of each segment breaking through the cylinder jacket in the radial direction of the endoscope, and/or is open radially outwards in the wedge back section of each segment (the cylinder jacket is broken open in the wedge back section as far as the working channel in the axial direction or has a longitudinal slit extending as far as the working channel), and wherein the number of segments in the wedge back section are connected to one another in the longitudinal direction of the endoscope by a single/common flexible, preferably elastic connecting plate, which is connected to the number of segments at the outer circumference of the latter only via a partial circumferential section (slightly larger than the slit width in the wedge back section) in order to close the working channel of each segment radially outwards, the connecting plate forming, on the one hand, a sliding surface for inserted medical instruments which runs continuously in the working channel (bridging the segments), and, on the other hand, hinge sections for the angular adjustment of two respectively adjacent segments.

The disclosure is based on the realization that the segments known from the prior art form edges and gaps between adjacent segments which extend transversely to the working channel. If the working channel is bent, an instrument which is pushed through the working channel is deflected in the working channel and slides off on an inner side of the segments (more precisely, on the side of the segments on which they form the joint contact), so that the instrument can easily abut against these edges and gaps and get caught. An additional tube that may be guided inside the working channel cannot solve this problem, especially in the folded down state. In summary, the inventive idea is based on the fact that in the area where the instrument mainly slides off, in particular in the area of the joint contact between the segments, a continuous, flexible slide-off plate (connecting element) or slide-off band device is provided which extends over (in particular all) the gap between adjacent segments of the fold-down mechanism and which forms a continuous slide-off surface, at least in the longitudinal direction of the endoscope, for the instrument as it is advanced through the working channel. This prevents an instrument from getting caught between the segments during advancement through the working channel.

In the area where the instrument mainly slides off, or in the area of the joint contact between the segments, the working channel wall formed by the segments is removed and the resulting opening is covered (preferably completely) by the slide-off band device/plate/connecting plate. This can also achieve further surprising effects. On the one hand, the diameter of the working channel can be increased due to the opening in the segments, thus creating more space for the required instruments or (with an unchanged working channel diameter) providing space in the segment cross-sections for further channels or components running through the endoscope. On the other hand, it is achieved that a hinge mechanism between the individual segments is particularly simple both in its structure and its manufacture/assembly and is therefore cost-effective.

In other words, the fold-down mechanism for a distal endoscope tip or being part of a distal endoscope tip (endoscope end portion) has a number of substantially cylindrical to oval segments (vertebral bodies) aligned with one another in the longitudinal direction of the endoscope, which are connected to one another at a partial circumferential section (the wedge back section) via a (common) flexible connecting plate/back element attached/fixed to the outside of the segments in such a way that they can be pivoted with respect to one another via the connecting plate acting as a hinge section. Corresponding hinge axes are each arranged between two segments. In particular, the flexible connecting plate acts as a film hinge at the hinge sections, i.e. between two segments in each case. Furthermore, the segments taper towards another partial circumferential section diametrically opposite the connecting plate (a wedge tip section) in such a way that a pivoting space/pivoting gap is provided between several segments, which makes it possible to actively shorten a length of the fold-down mechanism on this other side by a maximum axial length of the pivoting space, namely by pivoting the segments over the connecting plate/around the hinge axes formed thereby. Maximum folding preferably is achieved when the (all) segments abut the respective adjacent segments (or the proximally and/or distal endoscope section adjacent thereto)/when their end faces are in contact with each other. A working channel runs longitudinally through the segments and is arranged eccentrically to the segment cross-section in such a way that an outer circumference of the working channel and an outer circumference of the segment overlap each other in the wedge back section, so that an opening is formed at the corresponding overlapping area in the wedge back section along the entire segment in the longitudinal direction of the endoscope. This opening is closed by the connecting plate attached radially on the outside of the segments. The connecting plate thus represents a continuous, smooth, edge- and gap-free surface (i.e. the slide-off surface) running along the (entire) fold-down mechanism, against which an instrument pushed through the working channel can slide off evenly and is evenly redirected, in particular when the fold-down mechanism is in the folded-down state.

The segments can have a circular cylindrical or oval cross-section, for example. The end faces are preferably flat, but can also be concave also be concave or convex, for example. Furthermore, the fact that the working channel breaks through the cylinder jacket (the jacket surface) in a radial direction towards the wedge back section, i.e. the fact that the working channel is open at the side, enables a larger cross-section of the working channel, which simplifies the insertion of instruments. Optionally, a thin-walled inner tube can be provided, which runs through the working channel formed by the segments, in particular to seal it (especially between the segments).

The continuous sliding surface, which delimits the working channel in the wedge back section, is, in particular, a surface that is that is continuous (free of breakthroughs) and flat (free of edges and kinks) in an area where the instruments slide off when the fold-down mechanism is folded down/bent, as well as in a feed direction of the instruments. This makes it easy for inserted instruments to slide off and thus simplifies their insertion into the working channel.

Specifically, the endoscope according to the disclosure has an endoscope head which is preferably coupled via a manually activatable/bendable (endoscope) deflector to a preferably flexible (passively/automatically bendable) endoscope shaft, at the proximal end of which (facing towards the user/away from the patient's body) a handle is arranged/connectable. Between the endoscope head or the distal (facing away from the user/facing the patient's body) axial end section of the endoscope head and the endoscope shaft or preferably between the endoscope head or the distal axial end section of the endoscope head and the endoscope shaft or deflector, an (additional) fold-down mechanism (with a different/smaller fold-down radius compared to the deflector, if provided) is interposed, with at least one or more vortex-like segments, which are wedge-shaped when viewed from the side. Each segment is provided with at least one longitudinal through-opening (or a longitudinal through-hole), which forms an axial section of a (common) working channel. The longitudinal through-opening is offset radially outwards to the center axis of the respective segment in such a way that the diameter of the through-opening in the peripheral area of the segment extends beyond the outer circumference of the segment with maximum axial segment length due to the wedge shape, thereby creating a longitudinal slit or longitudinal gap. This preferably gives the segment a crescent-shaped contour in top view. Finally, a connecting plate/back element is provided which is adapted to the side circumference area with maximum axial segment length in such a way that when it is attached to the outside of the segment, it only covers this side circumferential area (i.e. not completely) and thus closes the longitudinal slot to the outside. Moreover, the connecting plate is attached to the outside of at least to the endoscope head or the distal head section and preferably to a further proximal segment of the same structure (if provided) and thus connects these segments to each other. In an axial section between the segments connected to thereto, the connecting plate is provided with a constriction in the circumferential direction, whereby a (folding) hinge is formed around which the segments connected thereto can be pivoted/folded down.

Advantageous embodiments of the disclosure are the subject of the sub-claims and are described in more detail below. These embodiments can be claimed in any combination or possibly also separately, i.e. in a separate manner in each case.

Preferably, the connecting plate covers the working channel in the wedge back section at least partially (namely in the area of the longitudinal slot) and is in particular curved over it.

The hinge sections advantageously have an extension in the longitudinal direction of the endoscope, in particular in such a way that the segments arranged or attached to the connecting plate are spaced apart. This increases the bending radius of the hinge sections and a transition between the folded-down segments is better rounded, i.e. more suitable for sliding off the instruments. In particular, an extension of each hinge section in the longitudinal direction of the endoscope can amount to 30 to 100% (preferably 40-70%) of an extension of the connecting plate in the area of the (adjacent) segments (i.e. in holding sections of the connecting plate to which the segments are held or attached).

Alternatively or additionally, the slide-off surface formed by the connecting plate in the area of the holding sections along the longitudinal direction of the endoscope can be curved/concavely rounded radially outwards. In other words, the slide-off surface forms (preferably only) in the area of the holding sections a trough or an (inside) arc that that rounds out radially in the longitudinal direction. If the thickness of the connecting plate is also constant in the curved area, a radial outer side of the connecting plate in the corresponding area (arch/bump-like) curves outwards along the longitudinal direction of the endoscope in the unfolded/stretched state of the fold-down mechanism. This can provide a slide-off surface which, in the folded-down state of the fold-down mechanism, i.e. when the instruments are deflected particularly strongly and generate a particularly strong radial force on the slide-off surface, is curved along its (entire) extension in the longitudinal direction of the endoscope, i.e. in the alignment direction, thus enabling particularly flat and stable sliding off.

Alternatively or additionally, the holding sections can curve radially outwards in the endoscope or segment circumferential direction over the working channel opening in the wedge back section. In particular, the curvature of each holding section essentially follows or approximates (e.g. polygonally) an outer diameter of the respective segment. The working channel is therefore not narrowed by the connecting plate and has plenty of space for instruments to be inserted. Furthermore, the curvature in the circumferential direction forms a guide channel, which guides inserted instruments in the longitudinal direction of the endoscope and prevents them from sliding back and forth.

Preferably, the connecting plate is scratch-resistant, at least in the area of the slide-off surface, in order to prevent the surface from being damaged by the instruments sliding off sliding off against it, which may make it difficult to disinfect. For example, the surface can be treated, e.g. hardened or coated, or the connecting plate can be made entirely or in the area of the slide-off surface from a scratch-resistant material, preferably a metal.

According to a preferred embodiment, the connecting plate preferably forms tab-shaped or ear-shaped holding sections, each of which serves to attach or hold it to one of the segments and/or to the distal end of the endoscope shaft/deflector. In particular, the holding sections and the hinge sections are arranged alternately to one another. The segments or the distal end of the endoscope shaft/deflector can be held securely on the holding sections. For example, the segments can be attached to the holding sections by gluing, welding or in a form-fitting manner. The shape of the holding sections moreover can be adapted to the segments to enable a particularly secure hold. particularly secure hold. At least one of the holding sections at a proximal end of the connecting plate is designed to attach/hold a distal end of the endoscope shaft or an endoscope deflector. More distal holding sections are preferably adapted to the segments of the fold-down mechanism.

It is particularly advantageous if the connecting plate is designed to be resilient at least at the hinge sections, optionally along its entire extension. The connecting plate (in particular the hinge sections) can thus serve as a return element which is designed to return the fold-down mechanism from a folded down position to an essentially forward/extended position (extending in the longitudinal axis of the endoscope straight or forming an obtuse angle thereto).

In other words, the connecting plate, at least in the area of the hinge sections, may be formed as a bending spring or leaf spring that preloads the fold-down mechanism into a substantially forwardly oriented/stretched position. The forward-oriented/stretched position is a position in which the endoscope head/a segment of the fold-down mechanism arranged furthest distally is oriented essentially forward, in a longitudinal direction of the endoscope or endoscope advance direction (if an endoscope deflector is provided, in an endoscope advance direction possibly predetermined by the deflector), such that an optical system provided therein enables a view to the front. With such a spring-elastic connecting plate, actuation by a user is only required to fold down the fold-down mechanism, as the return from the folded-down position is generated by the connecting plate. Thus, the number of actuating pulls can be reduced, for example, and use for the user is simplified.

Particularly preferred effects can be achieved if the spring elasticity of the connecting plate is variable along its longitudinal extension, in particular continuously decreasing or increasing. The spring elasticity can be achieved, for example, by treatment of the connecting plate, by providing weakening or strengthening structures through a changing material thickness or the like. The following example describes a specific design of the connecting plate, by which a spring elasticity of the connecting plate can be optionally adjusted along its axial extension.

It is also particularly advantageous if the hinge sections are curved in the circumferential direction (in particular radially outwards) such that each hinge section forms a groove running in the longitudinal direction of the endoscope with a specific, constant or variable, groove radius (radius of curvature/curvature/degree of curvature). Preferably, the groove radius/radius of curvature or the curvature/degree of curvature is adapted/selected depending on a desired restoring force. In other words, the restoring force of the (spring-elastic) hinge sections serving as restoring elements, among other things, is adjustable/set depending on their curvature along the circumferential direction. The restoring force is a force with which the hinge section (due to its spring elasticity) urges the fold-down mechanism back into the unfolded or stretched position. This means that the restoring force acts against the actuating element used for folding down and optionally also returns the actuating element to an initial position when it is no longer actuated by the user. The restoring force is stronger, the smaller the channel radius or the greater/tighter the curvature of the hinge section.

In functional terms, when the hinge sections bent or folded down, they are compressed at the groove edges/in the groove edge area are (elastically, possibly also plastically) and/or stretched in a central area/groove center area (viewed in the circumferential direction). However, since the groove edges/the curved hinge section edges counteract this compression (largely block it), they are deformed (especially elastically), or more precisely, pushed radially outwards. This means that they are deformed in such a way that the respective hinge section approaches (flattens) an essentially flat or straight (i.e. uncurved or unbent) shape along its hinge axis. More specifically, when the fold-down mechanism is folded down slightly, the hinge section initially flattens in the groove center section i.e. the hinge section is bent against the curvature in order to allow the hinge section to bend. If the fold-down mechanism is folded down more strongly/in the direction of the folded position, the essentially flattened area enlarges/widens in the circumferential direction, i.e. towards the groove edges, in order to enable the hinge section to be bent more strongly. The deformation resistance/elasticity of the material counteracts bending/folding down. In order to ensure a smooth fold-down of the fold-down mechanism, it is advantageous if the curvature/groove is mirror-symmetrical to a mirror plane running through the groove center and along the endoscope longitudinal axis of the endoscope. In other words, the course of the curvature/groove/bend starting from the groove center towards the groove edges is the same on both sides.

If the groove radius/the curvature (between the groove center and the groove edge) is variable, a restoring force can be set in particular, which varies depending on a fold-down/deflection radius (a degree of actuation) of the fold-down mechanism. In other words, the restoring force can be modified via the groove bend/the groove radius/curvature. In particular, the restoring force is set by groove/curvature radius and/or by a curvature curve (i.e. the change in the groove/curvature radius) along the endoscope/segment circumferential direction such that a smaller groove/radius of curvature corresponds to a larger restoring force or that a position of a set radius of curvature along the endoscope/segment circumferential direction corresponds to a fold-down angle/fold-down degree of the fold-down mechanism, whereby a maximum fold-down angle/fold-down degree corresponds to a position of the curvature at the groove edge and a minimum fold-down angle/fold-down degree corresponds to a position of the curvature in the center of the groove.

For example, the groove radius decreases towards the outer hinge section edges or groove edges in the circumferential direction. This means that the groove radius/radius of curvature is large or even not curved at all (i.e. straight) in the groove center area and small (i.e. strongly curved) in the groove edge area in order to ensure that a higher restoring force acts in the strongly bent state of the fold-down mechanism than in a less bent state. This can be advantageous, for example, as it provides the user with an improved sensory feedback on how much the fold-down mechanism is folded down. In other words, the groove radius/radius of curvature, viewed along the circumferential direction, can be comparatively large in the center of the hinge section and can decrease towards (lateral/free) hinge section edges or groove edges, or the curvature/degree of curvature can be comparatively small in the center and increase/become narrower towards the edges. If the groove radius decreases towards the edge or the curvature increases towards the edge, the restoring force is the greater the more the fold-down mechanism is folded down/bent.

Alternatively or additionally, the groove-/curvature-radius in the groove center area and/or in an intermediate area between the groove center and the groove edge can be curved more than along the rest of the groove cross-section. This means that a large restoring force can be can be provided in an only slightly (in particular minimally) folded-down state and/or a medium folded-down state. If the groove/hinge section is only comparatively slightly curved, or not curved at all in the groove/hinge section edge area the restoring force is low in the maximum folded-down state.

This is advantageous because in this case, in the maximum folded down/maximum actuated state of the fold-down mechanism, a user has to apply less actuating force. Simple (requiring little operating force) holding of the fold-down mechanism by a user in the maximum folded-down state is thus facilitated.

In summary, the restoring force can be increased (maximized) in the maximum folded down/bent state of the fold-down mechanism if the groove radius decreases (maximally) directly at the edge area and/or the restoring force can be increased (maximized) in an intermediate area, if the groove radius decreases (maximally) at an intermediate area (between the center and the edge of the groove) and/or the restoring force can be increased (maximized) in the only slightly (minimally) folded down/bent state (in the stretched state) of the fold-down mechanism, if the groove radius is (maximally) reduced in the groove center area.

The connecting plate can either be formed in such a way that each hinge section is the same, or the connecting plate can have several different, in particular differently curved, hinge sections. For example, the first hinge section located furthest proximally in the hinge/groove edge area can be curved more than in the remaining hinge/groove area, and the last hinge section located furthest distally in the hinge/groove center area can be more curved. The intermediate hinge sections along the longitudinal axis of the endoscope may each have a more strongly curved region that is slightly more central, starting from the first hinge section towards the last hinge section. In other words, the curvature is gradually moved further towards the groove center area along the longitudinal axis of the endoscope or, starting from the first hinge section with the strong curvature in the groove edge area, a gradual transition of the position of the strong curvature is provided up to the last hinge section with the strong curvature in the groove center area. This ensures that the fold-down mechanism folds down first at its proximal end/first at the most proximally located hinge section, then the other segments/the other hinge sections fold down one after the other in the longitudinal direction of the endoscope and finally the segment located furthest distally/endoscope head arranged thereon folds down or the fold-down mechanism folds down last at its distal end. The hinge sections can also be arranged in a different order, e.g. inversely to the order described above and thus fold down first at the distal end of the fold-down mechanism and fold down last at the proximal end.

It is particularly advantageous if the connecting plate (especially the hinge sections of the connecting plate) has a lower spring elasticity in the distal area than in the proximal area (especially in the area of the most proximal located segments of the fold-down mechanism or the deflector or in the area of the deflector or in the area of the most proximally located segments of the deflector). By this it is achieved that the endoscope rolls up starting from its distal end. Advantageously, this can reduce lateral swiveling of the distal endoscope head during bending. Thus, the present endoscope requires less space when folding down the endoscope head via the fold-down mechanism and possibly via the deflector. Preferably, the connecting element connects both the segments of the fold-down mechanism and of the deflector. Further preferably, a stiffness of the connecting element decreases along its axial extension in the distal direction, preferably continuously or uniformly.

Furthermore, it has proven to be useful if the connecting plate is tapered at the hinge sections (forming the aforementioned constrictions). This increases the flexibility/bendability of the hinge sections. Optionally, the hinge sections can still have sufficient torsional rigidity. Furthermore, the hinge sections should be wide enough to serve as part of the sliding surface.

Preferably, the connecting plate can form a smooth central section which is continuous in the longitudinal direction of the endoscope and forms the sliding surface from which tab-shaped fastening regions or tabs protrude symmetrically (at the holding sections) on both sides transversely or obliquely to the longitudinal direction of the endoscope or in the circumferential direction of the endoscope, which serve to fasten the segments and are bent towards each other with respect to the central section.

Preferably, the fastening areas can be wide in the longitudinal direction of the endoscope (in particular more than four times, preferably more than five times, more preferably more than ten times as wide) in relation to the slots or spaces formed between the respective adjacent fastening regions. Preferably the fastening regions essentially have a width in the longitudinal direction of the endoscope, which in each case corresponds to at least the maximum extension of one of the segments in the longitudinal direction of the endoscope, further preferably corresponds to the maximum extension of one of the segments in the longitudinal direction of the endoscope and a distance between two adjacent segments. The slits preferably may preferably be substantially linear/one-dimensional. In other words, the connecting plate may be provided with (parallel) slits on both sides, which separate each two adjacent fastening regions from each other. This allows the adjacent (sheet-like) fastening regions to slide over each other in an imbricate/scale-like when the fold-down mechanism is bent. This ensures that a gap between the segments/tilting bodies (at least in the wedge back area) is covered regardless of the curvature of the fold-down mechanism, even if the center area of the connecting plate is narrow. This even better prevents the instrument from catching during advancement through the working channel.

Additionally to the (first) connecting plate described above, a second connecting plate can be provided, which has a second central portion extending in the longitudinal direction of the endoscope and tabs projecting symmetrically therefrom transversely to the central section (in the circumferential direction of the endoscope). The second connecting plate is arranged, in particular, radially inside with respect to the endoscope (with respect to a mounted state of the fold-down mechanism) the first (radially outer) connecting plate. These two (first and second) connecting plates can be arranged so as to be displaceable/sliding relative to one another in the longitudinal direction of the endoscope, in particular in that one of the two connecting plates is only fixed at one point in the longitudinal direction of the endoscope relative to the other of the connecting plates and is otherwise freely displaceable.

Furthermore, the two connecting plates can be arranged offset in the longitudinal direction/placed on top of each other in such a way that the tabs of one connecting plate cover the spaces between the tabs of the other connecting plate at least partially, preferably completely. Preferably, the tabs of one connecting plate are at least as wide in the longitudinal direction of the endoscope as, and in particular wider than, the spaces between the tabs of the other connecting plate. The central sections of the first and second connecting plate are preferably located one above the other, and are also preferably located one above the other in the same position or congruently in the circumferential direction. This ensures that a gap between the segments/tilting bodies (at least in the wedge back area) is covered independent of the curvature of the fold-down mechanism. This even better prevents the instrument from getting caught during advancement through the working channel.

Further preferably, the segments (in particular all segments) are mounted on the first, radially outer connecting plate as described above. In this case, the tabs of the second, radially inner connecting plate serve to cover the spaces between the tabs of the radially inner connecting plate, thus forming cover areas. In other words, the second, radially inner connecting plate/cover plate inner connecting plate/cover plate (in short: plate), which covers the openings of the first plate, should lie between the first, radially outer plate and the segments/tilting elements. In particular, the second plate is fixed in position at only one point in the longitudinal direction of the endoscope relative to the first plate (i.e. attached to (exactly) one of the segments or (exactly) one of the vertebral bodies of the deflector or the endoscope head/distal head section and/or to the first plate). In particular, the second plate thus can move against the first plate and is nevertheless locked in, i.e. guided along the longitudinal direction of the endoscope in the fold-down mechanism both in the neutral and folded/bent state (in such a way that it cannot protrude radially outwards beyond the segments). Accordingly, when the fold-down mechanism is bent down, the tabs of the radially outer, second connecting plate can slide over the tabs of the radially inner, first connecting plate like scales.

Preferably, the tabs of the radially outer connecting plate are wider in the longitudinal direction of the endoscope than the tabs of the radially inner connecting plate. Further preferably, the tabs of the second, inner plate are narrower in the longitudinal direction of the endoscope than the distance between two adjacent segments, such that they do not collide with the segments when the fold-down mechanism is folded down.

Alternatively, the segments can be attached alternately to the first, radially inner and the second, radially outer connecting plate.

In particular, the first and second connecting plates are each only half as thick in their plate thickness/sheet thickness (i.e. viewed in the assembled state in the radial direction of the endoscope), compared to an arrangement in which a single connecting plate is provided.

In particular, the fold-down mechanism according to the disclosure is provided and configured for holding of the endoscope head at the distal end of an (endoscope) deflector. This makes it possible, on the one hand, to fold down the endoscope head strongly/tightly by the fold-down mechanism in order to allow a view to the rear or to the side even when examining very narrow body cavities of a patient and, on the other hand, to achieve a wide bending of the endoscope head by the deflector in order to provide safe navigation even in larger body cavities. In other words, preferably a bending radius of the fold-down mechanism when it is fully folded down is smaller than a bending radius of the endoscope deflector when it is fully bent down. The endoscope is preferably suitable or configured as a duodenoscope, wherein the backward-facing or lateral orientation of an optic of the endoscope head is provided by the fold-down mechanism.

Furthermore, it is advantageous if the endoscope according to the disclosure has a sheathing tube or endoscope tube (a so-called “overtube”/tube sleeve/sheathing; in short: tube), which is provided separately from the fold-down mechanism and in particular surrounds/encloses it in the circumferential direction (essentially completely). The endoscope tube serves to protect patient tissue from being injured by the fold-down mechanism, e.g. by pinching, when the endoscope is used. Furthermore, the endoscope tube prevents the segments (and possibly the endoscope head) from slipping or twisting in relation to each other around the hinge sections, i.e., the tube holds the segments (and possibly the endoscope head) in an aligned position. In similar systems, tensioning wires are usually used, which can also be dispensed with, thus reducing manufacturing costs, especially assembly costs. In other words, the tube forms an envelope around the fold-down mechanism, in particular one that is closed at least in the circumferential direction. Moreover, the tube can be closed at a distal end (facing the patient) and can be placed over the endoscope head or the last segment of the fold-down mechanism. Alternatively or additionally, the tube can be fixed to the endoscope head.

Preferably, a channel adjustment mechanism is provided, which makes it possible to increase and/or decrease the diameter of the working channel. This is advantageous since the working channel thus can be quickly and easily adapted for use with larger or smaller instruments without requiring additional tools or needing to produce segments of different sizes. This means that the number of segments to be manufactured is increased and the manufacturing costs are reduced accordingly, without sacrificing the variability of desired working channel diameters. In other words, a channel adjustment mechanism is provided which is configured to increase and/or decrease a working channel diameter, and which is provided in that the segments in the wedge back portion and the connecting plate are configured to be bendable radially inwards and/or outwards with respect to the longitudinal direction of the endoscope.

The channel adjustment mechanism is provided by the fact that segment wings of the segments which at least partially embrace the working channel (ends of the crescent shape of the segments/segment parts forming the wedge back section) are formed such that they can be elastically and/or plastically bent open radially outwards and/or bent closed radially inwards. In other words, the segment wings can be bent in such a way that their tips can be bent away from each other or towards each other.

Furthermore, the connecting plate (back element) preferably has channel adjustment structures, which locally reduce the bending stiffness of the connecting plate such that the connecting plate is configured to be bendable open and/or bendable closed in a defined radially inward and/or outward direction with respect to the longitudinal direction of the endoscope. In other words, the connecting plate (also referred to as the back element) has channel adjustment structures at least at the holding sections (in particular at a transition to between the respective fastening and cover regions or a part of the fastening region facing the cover regions), optionally also at the cover regions and/or on the hinge sections. Preferably, the channel adjustment structures are provided exclusively at the fastening regions, in particular only at a transition to the respective cover regions. Each holding section of the connecting plate is provided with at least one channel adjustment structure. The channel adjustment structures enable or simplify the bending of the holding sections to open and close, and possibly also the cover and hinge sections (of the central section), radially outwards or inwards with respect to the longitudinal axis of the endoscope. This means that the channel adjustment structures represent target bending points that allow defined, simple bending of the connecting element, in particular of its fastening regions relative to its cover areas/central section, in order to adjust its curvature in the circumferential direction of the endoscope.

The channel adjustment structures can be any structures that define and locally reduce the bending stiffness of the back element. Preferably, identical channel adjustment structures are (exclusively) provided along the connecting plate. However, it is also conceivable to provide different channel adjustment structures on different holding and/or cover and/or hinge sections. In particular, it is advantageous to provide different channel adjustment structures at the most proximal and/or most distal holding sections that are used for attachment to the endoscope shaft or -deflector or -head. It is also conceivable to provide different channel adjustment structures at the fastening regions than at the central section.

Preferably, the channel adjustment structures (all or part of them) are provided via slots in the connecting plate that are thin enough not to interfere with sliding of the surgical instrument on the back shell, even if the slots are provided in the cover areas and hinge sections. A slot penetrates the connecting plate and opens in both a radially outer and inner connecting plate surface. Each holding section or each fastening region of the connecting plate may be provided with at least one slot as a channel adjustment structure. Preferably, a grid of slots is provided as a channel adjustment structure. Preferably, the slots are V-shaped and oriented such that a tip of the V-shape points outwards or inwards in the circumferential direction of the endoscope. Alternatively, the slots can run in a straight line in the longitudinal direction of the endoscope and can be arranged in succession in the manner of a perforation, so that the bending stiffness of the connecting plate is reduced along the perforation and a longitudinal extension of the perforation defines the bending axis. Alternatively, the slots can run in the circumferential direction of the endoscope so that at least one portion of the holding sections/the fastening regions is lamellar or grid-like. Furthermore, diagonal slits can be provided.

Alternatively or additionally, the channel adjustment structures (all or part of them) can be provided via variations of a cross-sectional profile of the connecting plate. For example, the material thickness of the connecting plate may be reduced in fold-like manner so that one or more groove(s) is/are provided, in particular in such a way that these extend(s) in the longitudinal direction of the endoscope over each holding section/each fastening region. The respective grooves can be provided only on a radial outer side or only on a radial inner side of the connecting plate. Alternatively, the respective grooves can be provided both radially on the inside and radially on the outside in such a way that they are opposite each other or that they are offset from each other (in particular in the circumferential direction of the endoscope). Similar to the slots, the grooves can also run in the longitudinal direction of the endoscope or in the circumferential direction of the endoscope or diagonally thereto. Alternatively or additionally, a cross-sectional profile of one of the holding sections, in particular at the transition between the fastening and cover regions, can be corrugated in order to provide a channel inserting structure. The at least one corrugation extends in the longitudinal direction of the endoscope over the entire holding section/fastening region, so that the bending stiffness of the holding section/fastening region is reduced along the corrugation, with the longitudinal extension of the corrugation defining the bending axis.

Alternatively or additionally, the channel adjustment structure(s) may be a section with reduced material strength relative to the rest of the connecting plate, so that the bending stiffness is reduced in this area. For example, this section is provided at a transition between the fastening regions and the cover regions. Preferably, one or more sections with reduced material strength extending in the longitudinal direction of the endoscope are provided at each holding section/fastening region. These may be made, for example, of a different material or may be provided through material treatment. If necessary, the entire fastening regions can be made of a material with lower bending stiffness than the hinge sections or the entire central section of the connecting plate. For example, the fastening regions can be made of a plastic and at least the hinge sections, preferably also the cover regions (i.e. the entire central section), can be made of a spring steel.

According to a further advantageous embodiment of the disclosure, the segment is divided into two (preferably separate) partial segments, which are attached to the connecting plate and connected to each other via tab-like fastening regions of the connecting plate. A (radial) gap is formed between the partial segments on a side of the cylinder jacket of the segment facing away from the working channel side (in particular the wedge tip section). The connecting plate/the fastening regions act(s) as an expansion spring, which spreads the two partial segments in order to widen the gap (so that an angle opens up between the partial segments) to provide a space for an additional working channel within the spread gap. The fastening regions at least partially surround the partial segments and, in a delivery state of the endoscope, are held via a locking element in an elastically inwardly deformed state, in which the partial segments are positioned close to one another, in particular essentially abutting one another (possibly with an intermediate lining or an intermediate additional tube, as described in more detail below). In this state, the expansion spring is pre-tensioned and the gap is essentially closed. When the locking element is removed, the gap is opened by the pretension of the expansion spring. The latch element can be a cord-like element such as a latch wire, which is threaded through eyelets in the fastening regions and can be pulled off in a proximal direction if an additional working channel is required. Alternative latch elements, such as e.g. a removable compression tube or similar are conceivable.

The additional working channel or the gap is preferably lined with a tubular film, which lies folded in the gap when the room/additional working channel is closed and runs through the fold-down mechanism in the longitudinal direction of the endoscope in order to seal the gap. The lining is preferably made of a thin EPTFE or TPU material. When the gap opened, the lining folds open to bridge/close the gap at the wedge tip section. Alternatively or additionally, an elastic additional tube can be provided, which is arranged between the partial segments in the slot (preferably glued to both sides of the partial segments) in such a way that it is unfolded by spreading the partial segments to serve as an additional working channel. A removable film or the like can preferably be provided in the additional tube or inside the lining to prevent the tube or lining state from sticking together when closed.

Preferably, each segment is divided into two separate halves in mirror symmetry with respect to a plane passing through the working channel and the longitudinal axis of the endoscope, and is accordingly formed by two (essentially semi-cylindrical) partial segments in such a way that each partial segment forms a partial cylinder jacket. In this case, each partial segment has a recess at the wedge back section in such a way that the recesses of the two partial segments arranged next to each other are opposite each other and together form the working channel (i.e. each partial segment forms a partial circumferential wall of the working channel). Radially outer sections of the recesses are spaced apart from each other so that in the area of the wedge back section, the cylinder jacket is pierced by the recesses or the working channel formed by those recesses.

Alternatively, the task underlying the disclosure is solved by a fold-down mechanism and in particular by a (medical) endoscope including the features of one of the independent claims.

More specifically, an endoscope is provided having an endoscope shaft with a distal, manually operable fold-down mechanism for holding an endoscope head or distal head section, which can be folded down or bent in a controlled manner, on an endoscope section located proximally upstream of the fold-down mechanism. The fold-down mechanism has a number of essentially cylindrical segments which follow one another axially and can be actively angularly adjusted relative to one another by means of at least one common actuating element, which segments each have two axial end faces aligned essentially in a wedge shape relative to one another and a cylinder jacket which defines a wedge back section at a section of the cylinder jacket with maximum axial length, at which the number of segments hinged relative to one another (or to the endoscope head/head section and/or to the proximally upstream endoscope section), and which each have at least one through-opening in the longitudinal direction of the endoscope such that the number of segments jointly form a working channel (each segment having a working channel wall section which is circumferentially continuous or closed). A flexible slide-off band/slid-off band device/slid-off plate is inserted into the working channel, extending in the longitudinal direction of the endoscope along the number of (in particular several) segments, preferably along the entire fold-down mechanism, in such a way that the slide-off band/slide-off band device/slide-off plate (at least partially) covers a wedge-back section of a working channel wall of the working channel (i.e., the working channel wall section of the respective segments) (and an area provided between the segments, e.g. gaps/edges/angles) and forms a continuous, smooth sliding surface for medical instruments inserted into the working channel.

In other words, the fold-down mechanism corresponds in its basic structure to the fold-down mechanism according to the above-described solution, comprising cylindrical segments arranged along the longitudinal direction of the endoscope with respect to each other, which are wedge-shaped in a side view and which are hinged to each other at the wedge back section in order to be angularly adjustable/foldable with respect to each other via an operation of an actuating element. such that a gap between the wedge tip sections of the segments is closed when folded down. Each segment has a through-opening/bore with a continuous/circumferential/closed circumferential wall (working channel wall section), wherein the through-openings of the segments jointly define a working channel. A flexible, preferably elastic, elongated rectangular slide-off plate/band device is inserted into the working channel on the wedge back side (on a side facing the wedge back section/on the side where the segments are hinged to each other) and extends through several, in particular all segments of the slide-off mechanism as well as over gaps or edges/angles formed between the segments. In summary, the segments are connected to each other via joints formed separately from the slide-off plate/band device and the slide-off plate/band device covers the joints.

When the fold-down mechanism is bent, the segments are folded down towards each other such that the wedge tip sections are moved towards each other, and possibly come into contact with each other. This means that the working channel wall sections of adjacent segments are also at an angle to each other, possibly with additional gaps between the segments, especially when not completely folded down. The slide-off plate/band device bridges the angles and any gaps and curves over them, so that the slide-off plate/band device provides a smooth, rounded sliding surface on the outer radius of curvature (along the wedge back sections of the segments that are folded down towards each other). The slide-off plate/band device has a hardness or the sliding surface is hardened in such a way that a uniform, smooth sliding of the instrument is made possible (without the sliding surface being substantially damaged, i.e, beyond superficial longitudinal scratches).

In other words, the slide-off plate/slide-off band device forms a stiffening element which is inserted into the working channel (in particular between the inner tube of the working channel and the inner wall of the segments/tilting bodies) at a region on the wedge back side.

Preferably, the slide-off band device is fixed to a distal end region of the fold-down mechanism, in particular (exclusively) on the endoscope head or distal head section or to a (single) segment arranged furthest distally (optionally instead to any other (single) segment, in particular to the first, second or third segment counted from the distal head section or endoscope head), for example, by gluing/welding/soldering (rotationally and axially fixed/without degrees of freedom).

Further preferably, the slide-off band device lies slidably in the longitudinal direction of the endoscope in the through-openings of a number of more proximally arranged (or, in the case of attachment to another segment, in the distally and/or proximally arranged) segments (preferably in all others) such that the slide-off band device can slip in the working channel during bending of the fold-down mechanism in order to substantially avoid stretching or compression of the slide-off band device in the longitudinal direction of the endoscope. In other words, the slide-off band device is attached to a (single) element (consisting of the segments and the distal head portion or the endoscope head) of the fold-down mechanism and runs loosely through the other elements, so that the slide-off plate/band device moves in a length-compensating manner in the longitudinal direction even if the length of the working channel changes due to the folding-down of the fold-down mechanism and does not corrugate, i.e. remains straight and smooth.

If the slide-off band device/slide-off plate is fixed in position at the fixing point and otherwise lies loosely in the working channel, the slide-off plate/slide-off band device, during folding of the fold-down mechanism curves, substantially following the curvature of the fold-down mechanism in such a way that it merely touches the walls (several or all) of the through-openings (i.e. the working channel wall sections formed by the individual segments). Thus, in the folded-down state of the fold-down mechanism, an angular difference and/or gaps are bridged by the slide-off plate/band device, which thus provides a smooth, (essentially uniformly) curved sliding surface for the instrument as it is advanced through the working channel.

Furthermore, it is advantageous if a proximal (loose/unattached) end of the slide-off plate/band device protrudes in the longitudinal direction of the endoscope into the endoscope section proximally upstream of the fold-down mechanism in a sliding manner. This ensures that the loose end of the slide-off band device/slide-off plate cannot get caught between segments and, for example, blocks or damage the fold-down mechanism. This means that the loose end is securely inserted into the working channel.

Furthermore, it is preferred if the slide-off band device is elastic and extends essentially straight in a rest position, so that the slide-off band device strives to return to the straight, unbent rest position when the fold-down mechanism is reset from a folded-down position to an essentially forward-oriented or stretched position. In particular, it is advantageous if the slide-off band device/the slide-off plate is rigid in the longitudinal direction of the endoscope such that forces acting on the slide-off plate/slide-off band device during use of the fold-down mechanism always lead to large-radius curvatures of the slide-off plate and, for example, local wave formation or displacement or twisting of the slide-off band device in the working channel are avoided. It is also preferable if the slide-off band device is essentially inextensible in the longitudinal direction of the endoscope.

Preferably, the slide-off band device as a whole is in the shape of a band/a plate (i.e. a slide-off plate) and forms a (particularly rectangular) closed surface free of openings.

Alternatively, preferably, the slide-off band device can have several longitudinal elements which extend in the longitudinal direction of the endoscope, in particular in parallel to each other. The longitudinal elements can be in the form of longitudinal strips, between which longitudinal slots are provided. In particular, the longitudinal strips can move relative to each other. Accordingly, different (longitudinal) displacements of the longitudinal strips can be provided across the width of the slide-off band device (i.e. depending on the circumferential position of the longitudinal strips on the fold-down mechanism in relation to the joints between the segments) in order to advantageously compensate for the fact that the fold-down mechanism is not shortened on a wedge back side of the segments (in the area of the joints), but is greatly shortened on a wedge tip side. For example, the slide-off band device can be formed in the shape of a fork, i.e. it can have several prong-like longitudinal strips which are connected at their distal end via a cross web. At the cross web, the slide-off band device can be attached to a distal area of the fold-down mechanism or to the endoscope head in such a way that the longitudinal strips extend in a proximal direction through the fold-down mechanism. In this case, the longitudinal strips are arranged in the working channel (between the segment inner wall/working channel wall of the segments and an inner tube of the working channel) along the fold-down mechanism so that they can slide freely/longitudinally to the segments.

Alternatively or additionally preferred, the slide-off band device may have several wires running in the longitudinal direction of the endoscope as longitudinal elements. The wires are inserted into the working channel (between the inner tube and the segment inner wall/working channel wall of the segments) and are optionally guided in grooves on the working channel wall. The wires can be attached at their distal end regions, in particular their distal ends, to the distal region of the fold-down mechanism or to the endoscope head and otherwise slide freely sliding in the working channel. This makes it particularly easy to enable the different displacements of the slide-off band device across the width of the slide-off band device.

In particular, the solution described above can be modified by providing the endoscope, in addition to the connecting plate/back element, with a slide-off band device as described above. This means that, on the one hand, the segments in the wedge back section are connected to one another in the longitudinal direction of the endoscope by a flexible, preferably elastic, connecting plate/back element, which is fixed to the respective outer side of the segment while covering the respective longitudinal slots. On the other hand, a flexible slide-off band device (can also be regarded as an additional back element) extending in the longitudinal direction of the endoscope along several segments, preferably along the entire fold-down mechanism, is inserted into the working channel such that it at least partially covers the connecting plate and/or the respective segments in the area of the wedge back section.

In particular, the slide-off band device is arranged at a transition area between the segments and the connecting plate and preferably covers the transition area. Advantageously, the slide-off band device can be arranged directly at an end (viewed in the circumferential direction) of the segments that is furthest from the wedge back side of the segments (i.e. in extension of the crescent-shaped contour/the sickle tips of the segments). In other words, a slide-off band device can be inserted into the working channel as one or more elastic reinforcing profiles. In particular, the slide-off band device can be inserted into at least one free space/space between the inner tube of the working channel and the wedge back section of the tilting bodies/segments (in the case of a closed working channel) or the connecting plate (in the case of a working channel that breaks through the cylinder wall of the segment on the wedge back side).

In the following, a further preferred embodiment of the present disclosure, which may be claimed independently, is described. This relates to an endoscope having a deflector including a plurality of (in particular diamond-shaped or wedge-shaped) vertebral bodies hinged to one another in a tiltable manner (in particular as described above) and an endoscope shaft arranged proximally to the deflector. The endoscope has an actuating mechanism with an actuating spring/coil spring (in short: spring), the distal end of which is fixed to a distal area, in particular to a vertebral body of the deflector arranged furthest distally, extends through the deflector and the endoscope shaft, and can be actuated in the longitudinal direction of the endoscope in order to control bending of the deflector.

A distal spring area of the actuating spring has a greater pitch (distance between two coils of the spring) than a proximal spring area in which the coils preferably lie against each other. In particular, a transition area between the proximal and the distal spring area is located in the area of the deflector when viewed along the length of the endoscope. A locking wire is attached to the distal end of the spring or of the distal spring region, which extends through the deflector and the endoscope shaft and can be actuated in the longitudinal direction of the endoscope. Preferably, the locking wire is guided within the spring. Preferably, the distal spring region is pretensioned by the locking wire in a neutral position (unactuated by the actuating mechanism/not bent) of the distal endoscope region (comprising at least the deflector), which advantageously increases the stability of the actuating mechanism/the distal end of the endoscope.

When the locking wire is actuated (pulled) in the proximal direction in a first actuation area, the distal spring area is initially compressed until the spring blocks (the coils of the spring come into contact in the distal spring area). As a result, the most distally arranged vertebral bodies of the deflector, which are arranged in particular distally from the transition area between the proximal and distal spring area, are bent/folded down relative to each other. The locking wire can be actuated in the proximal direction in the first actuation area. The actuating spring is preferably supported at a proximal end by a support spring (compression spring) having a predetermined stiffness which is configured to minimize or prevent movement of the actuating in the proximal direction during actuation in the first actuating region of the locking wire.

If the locking wire and/or the (blocked) actuating spring is further actuated/pulled in the proximal direction after actuation in the first actuation area, actuation is provided in a second actuation area. In this case, the locking wire moves together with the actuating spring (possibly against the support spring/compressing the support spring) in the proximal direction in order to also bend the proximally arranged vertebral bodies of the deflector, which are located in particular proximally to the transition area. This allows the deflector, which is used to control/navigate the endoscope in a patient cavity, to be bent in multi-stage actuation ranges, which enables particularly sensitive control.

Furthermore, the endoscope may comprise a distal fold-down mechanism/tilting head (in particular as described above) having a plurality of wedge-shaped segments hinged to each other in a tilting manner, which is arranged distally to the deflector and can be bent at a smaller angle than the deflector. The actuating mechanism can have a control wire (push-pull wire/Bowden cable) which is fixed to the distal area of the fold-down mechanism or an element arranged distally thereto (e.g. an endoscope head), extends through the fold-down mechanism, the deflector and the endoscope shaft, and can be actuated in the longitudinal direction of the endoscope, in order to control bending of the fold-down mechanism. In particular, the control wire is guided through the actuating spring. Thus, the control wire and the actuating spring can be optimally pretensioned against each other/work against each other, which increases stability of the actuating mechanism/the distal endoscope (comprising at least the deflector and the fold-down mechanism).

In the neutral position of the distal endoscope region, the deflector is preferably unactuated/unbent by the actuating mechanism and the actuating spring is pretensioned by the locking wire, as described above. If the control wire is actuated (pulled) in the proximal direction starting from the neutral position of the distal endoscope region, the segments of the fold-down mechanism are bent relative to each other. Thus, for example, bending of the fold-down mechanism by 120° to the longitudinal axis of the endoscope/to the endoscope section immediately proximal to it (the deflector) can be achieved. In particular, the fold-down mechanism achieves a particularly small radius of curvature and is therefore particularly suitable for enabling lateral or retrograde alignment of an endoscope head/distal head section in a narrow space.

The pretension of the distal spring area of the actuating spring and, if necessary, a pretension of the support spring is preferably configured/adjusted in such a way that bending of the deflector is minimized or prevented by actuation of the control wire. Thus, actuation of the fold-down mechanism can be reliably decoupled from the actuation of the deflector.

Preferably, the control wire as well as the locking wire and possibly the actuating spring are motor-driven in order to be able to actuate them independently of each other in the longitudinal direction. If the deflector is actuated in the first or second actuation range, the control wire is preferably actuated at the same time to actuate the fold-down mechanism in order to compensate for a change in the length of the control wire caused by the bending of the deflector in order to maintain the position of the fold-down mechanism. Alternatively, the control wire can be actuated to set a desired curvature of, for example, up to 180° of the distal endoscope region as a whole (i.e. the fold-down mechanism and the deflector). Thus, for control/navigation of the endoscope in the patient cavity, the fold-down mechanism/tilting head and the deflector can be used together and the fold-down mechanism does not constitute an awkward, lunging obstacle when steering around curves, for example.

The motor control/motor drive also makes it possible, for example, for the tilting head elements (i.e. the segments of the fold-down mechanism) to be bent in parallel with the bending of the most distally arranged deflector elements (i.e. the corresponding vertebral bodies of the deflector). With suitable control, the tilting head elements can be controlled to the same bending radius as the deflector elements, resulting in a uniform bending radius.

Preferably, the vertebral bodies of the deflector are connected to each other by a further deflector connecting/back element (back armor/connecting plate), which is essentially of the same design as the connecting/back element (back armor/connecting plate), via which the segments of the fold-down mechanism are connected to each other, wherein dimensions of the deflector connecting/back element (in particular an extension of the holding or fastening sections in the longitudinal and/or circumferential direction of the endoscope and/or a distance between the holding or fastening sections) are adapted to the vertebral bodies of the deflector. The deflecting connecting/back element may be integrally formed with the connecting/back element connecting the segments of the fold-down mechanism. In other words, the deflector may also have a back spring/connecting/back element. Both areas (i.e. the fold-down mechanism and the deflector) may be covered with a (in particular single/common) spring (back spring/connecting/back element) (i.e. at the wedge back area), which has a different pitch (in particular between holding and hinge areas) in the tilting head area and deflecting area.

In particular, the vertebral bodies of the deflector are wedge-shaped in a side view and cylindrical in a top view, but are longer in the longitudinal direction than the segments of the fold-down mechanism. A wedge back section of the deflector can be arranged at the same circumferential position as the wedge back section of the segments of the fold-down mechanism. The wedge back sections of the deflector (on the vertebral body side) can be connected/hinged to each other by the deflector connecting/back element. If necessary, a second connecting/back element as already described above can also be provided which is offset in the longitudinal direction to the deflector connecting/back element and which is arranged radially inwards offset to the connecting/back element of the fold-down mechanism and/or to the deflector.

According to an aspect of the present application which may be claimed independently, a control method for controlling an endoscope with a distal endoscope head, a fold-down mechanism arranged proximally thereto for folding down the endoscope head, and a deflector arranged proximally to the fold-down mechanism, in particular of an endoscope described above, is provided, wherein at least the fold-down mechanism can be driven independently of the deflector.

The control method can preferably switch the endoscope to a first mode in which only the fold-down mechanism can be folded down or is folded down independently of the deflector. Further preferably, the control method can switch the endoscope to a second mode in which the deflector can be folded down or is folded down, e.g. after a specific, in particular completely folded down, position of the fold-down mechanism has been reached by control in the first mode.

Further preferably, the control method can switch the endoscope to a third mode in which the fold-down mechanism and the deflector are curved or folded down, in particular substantially simultaneously, further in particular with a substantially constant radius of curvature along the fold-down mechanism and the deflector, or can be curved or folded down.

Further preferably, the control method can switch the endoscope to a fourth mode in which the fold-down mechanism can be further folded down or is folded down, e.g. after the deflector has reached a specific, in particular maximum, curvature through the control in the third mode. Ultimately, however, it is also possible to bend the deflector and/or the fold-down mechanism within the scope of their respective geometric possibilities, preferably alternately in opposite directions, in order to realize an S or inverted S shape (i.e. the deflector and/or the fold-down mechanism can be overstretched or bent radially towards the circumferential side to which the back element is attached).

Preferably, the control method can retrieve or receive data about a position of the endoscope head within a patient body. This data can originate from a position detection means such as a sensor on the endoscope head, a stored treatment planning, a sensor system or the like, which are preferably part of a treatment system with the endoscope. Further preferably, the control method can switch the endoscope to the first, second, third or fourth mode depending on the determined data about a position of the endoscope in the patient's body. For example, the control method can operate the endoscope in the first mode if the endoscope head is located in the small intestine near the papilla or if it is located in the large intestine. In this way, a targeted and simple backward view can be provided to search for the papilla or for intestinal polyps or the like. Further, for example, the control method can operate the endoscope in the third mode when the endoscope head is in the stomach. Therein is plenty of space for navigation and the steering of the endoscope can therefore be optimized. If it is determined that the endoscope head reaches an end region of the stomach, the control method can, for example, switch the endoscope to the fourth mode. This can make it easier to find an exit of the stomach and the endoscope can be steered more precisely to move to/through the stomach exit.

Moreover, the endoscope can have at least one drive for driving the deflector and one drive for driving the fold-down mechanism. The control device can be configured to drive the drives at different speeds and independently of each other, depending on the mode. In other words, the control method controls the different modes in particular by controlling the drives, e.g. electric motors or stepper motors, differently, i.e. operating them at different speeds depending on the mode. In this way, the influence of a position/movement of one of the deflectors and the fold-down mechanism on the control of the respectively other one can be compensated for.

Moreover, a further aspect of the present application, which may be claimed independently, relates to a treatment system with an endoscope described above (in particular with regard to the control method), and a computer unit or control device which is configured to carry out the above control method and which is set up in particular to retrieve and process the data on the position of the endoscope head and/or to control drives for driving the deflector and the fold-down mechanism via the control wires.

### BRIEF DESCRIPTION OF THE DRAWING FIGURES

In the following, the present disclosure is described by means of preferred embodiments. However, these are only illustrative in nature and are not intended to limit the scope of protection of the present disclosure. Furthermore, identical reference signs are used for identical components in the description of the various embodiments.

FIG. 1 shows a perspective view of an endoscope head of the disclosed endoscope according to a preferred embodiment in a bent and straight position.

FIG. 2 shows a top view of the endoscope head of the preferred embodiment.

FIG. 3 shows a back element of the endoscope according to a first preferred embodiment and a modification of the back element.

FIG. 4 shows the back element of the first embodiment in a perspective view.

FIG. 5 shows a cross-sectional view of the back element through a hinge section curved in a groove-like manner according to a modification of the first embodiment.

FIG. 6 shows a top view of the endoscope head to illustrate the functional principle of a channel adjustment mechanism of the first embodiment.

FIG. 7 and FIG. 8 show the back element of the preferred embodiment with channel adjustment structures of the channel adjustment mechanism according to several modifications.

FIG. 9 and FIG. 10 show a cross-section through the fold-down mechanism according to a further embodiment.

FIG. 11 shows the side view of a fold-down mechanism according to an alternative design of the disclosure in the stretched and unfolded state.

FIG. 12 and FIG. 13 show two preferred embodiments of the alternative design.

FIG. 14 schematically constitutes a further preferred embodiment of the fold-down mechanism according to the present disclosure.

FIG. 15 illustrates one aspect of a modification of the embodiment according to any of FIGS. 1 to 8.

FIG. 16A to FIG. 16 C show an actuation sequence of an actuating mechanism for actuating an endoscope according to one of the embodiments of FIGS. 1 to 15.

FIG. 17 schematically shows a modification of the endoscope according to FIG. 1.

FIG. 18 shows an endoscope, in particular according to one of the above figures, in various adjustable operating states.

### DETAILED DESCRIPTION OF THE PREFERRED EMBODIMENTS

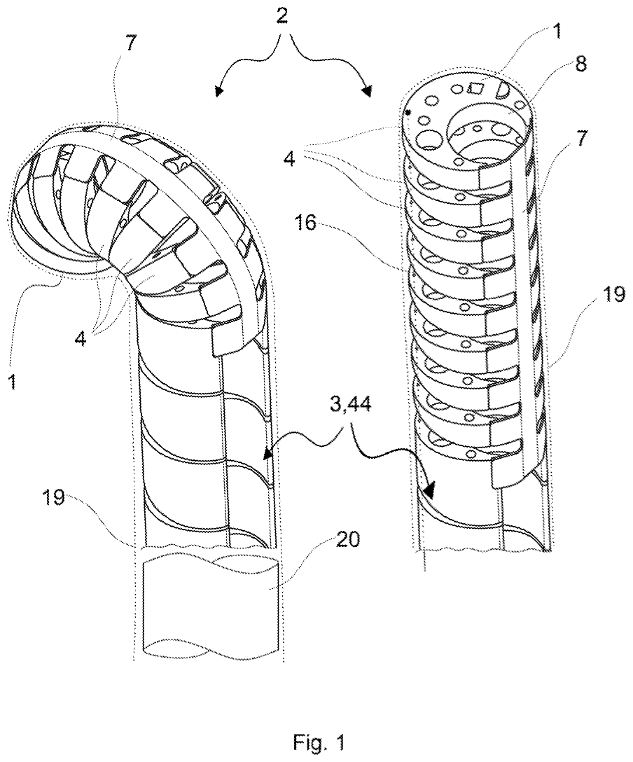

FIG. 1 is a perspective view of an endoscope head 1 of the endoscope according to the disclosure according to a preferred embodiment. The endoscope head 1 has a fold-down mechanism 2 (as a component of the endoscope head 1 itself or as a separate unit for this purpose), which is shown bent/folded down in FIG. 1 on the left and stretched out on the right in FIG. 1. In particular, the endoscope is a duodenoscope and the fold-down mechanism 2 is preferably (but not necessarily) attached to the distal end of a deflector section 3 of the endoscope. The deflector section 3 is a flexible section that can be manually (controlled) actuated/deflected independently of the fold-down mechanism 2, through which the endoscope head 1 is preferably pivotable (i.e. can be tilted with a large bending radius with respect to the fold-down mechanism 2) for navigation of the endoscope. The deflector section 3—if provided—is fixed to the distal end of a preferably bendable endoscope shaft 20. In this case, the endoscope shaft 20 can be bent passively, i.e. cannot be manually activated/controlled, for example to follow bends in a colon. Furthermore, an endoscope tube or overtube 19, shown here in dashed lines, is provided, which is pushed over the fold-down mechanism 2 and, in particular, fixed to the endoscope head 1 in order to protect patient tissue from injury (e.g. from being pinched) by the mechanics of the fold-down mechanism 2 and/or the endoscope when the corresponding endoscope is used. The endoscope tube or overtube 19 is provided separately from the fold-down mechanism 2 and (fully) surrounding it in the circumferential direction.

The fold-down mechanism 2 can be folded down separately with respect to the deflector section 3 (with a small bending radius of the fold-down mechanism 2 with respect to the deflector section 3) in order to tilt at least a distal end section of the endoscope head 1 (including optics and working channel outlet) or the entire endoscope head 1 in a radial direction and thus enable a lateral (radial) or rearward view of an optics arranged on the endoscope head at the tip of the fold-down mechanism 2.

For this purpose, the fold-down mechanism 2 has a number of (vertebral body-like) segments 4 (at least one segment), which are essentially cylindrical to oval in shape, with consequently round or oval axial end faces, which face the end faces of adjacent segments 4 in a longitudinal direction of the endoscope, or face the endoscope head arranged distally to the fold-down mechanism or the distal head section and the deflector/endoscope shaft arranged proximally to the fold-down mechanism. In a longitudinal section along the longitudinal direction of the endoscope, each segment 4 is wedge-shaped (wedge-shaped disk), i.e. the end faces of the respective segment 4 are aligned at an acute angle to one another. The wedge-shaped segments 4 are arranged in a circumferential direction in relation to each other in such a way that wedge tip sections 5 of the segments 4 (i.e., lateral surfaces which have a minimum expansion/length in the longitudinal direction of the endoscope due to the wedge shape) are each located at the same position in the circumferential direction/circumferential position. Accordingly, the circumferential sections of the segments 4 with maximum axial length (hereinafter referred to as wedge back section/area 6) are also axially close to each other in the same circumferential position, such that in the non-actuated state of the fold-down mechanism 2 (prograde alignment in the design position), the wedge-shaped segments 4 project essentially at right angles to the endoscope axis, comparable to the teeth of a toothed rack.

If the fold-down mechanism 2 configured in this way is actuated (for example by way of a pull rope placed in the wedge tip section 5, not shown further), the wedge tip sections 5 of the segments 4 are moved towards each other (axially), so that a length of the endoscope head 1 is shortened on the side of the wedge tip sections 5, whereas the segments in the opposite wedge back section 6 support themselves against each other (directly or indirectly), thereby generating the fold-down.

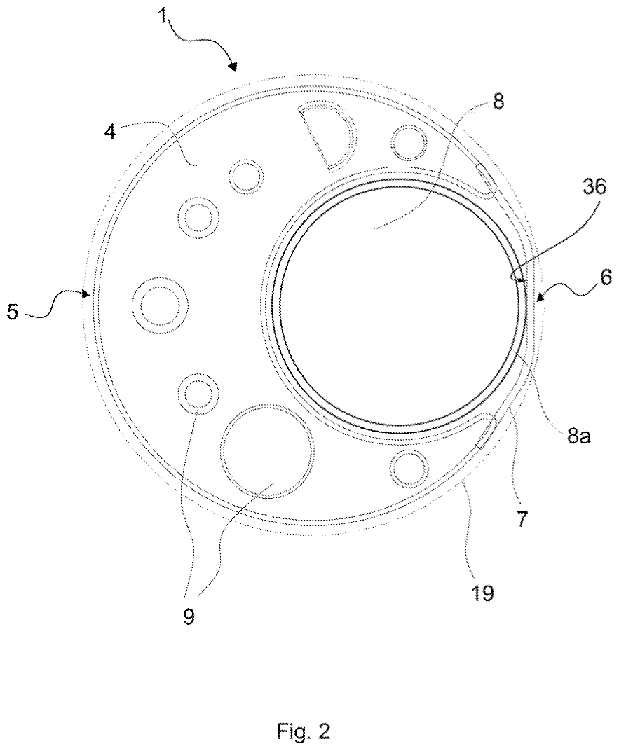

At the wedge back section 6 of the segments 4 opposite the respective wedge tip section 5 (i.e., lateral surfaces which have a maximum axial extension in the longitudinal direction of the endoscope due to the wedge shape), the segments 4 are connected to each other by a connecting plate/back element 7 (back armor/connecting plate). The back element 7 is a flexible, in particular elastic (thin-walled) connecting plate 7, preferably made of a metallic material, formed separately from the segments 4. FIG. 2 shows a top view of the endoscope head 1 or of the tip of the fold-down mechanism 2. The endoscope or each segment 4 has a working channel section 8 such that the working channel sections 8 of the segments 4 as a whole define a working channel that opens longitudinally in the form of a slot towards the wedge back section 6. In other words, the lateral surface of the/each segment 4 has a slit or gap-shaped opening in the radial direction at the circumferential position of the wedge back section 6, which extends axially over the entire length of the segment. The back element 7 extends or curves over the aperture and is fixed to the outer circumference of the segment 4 on both sides thereof in the circumferential direction, without fully enclosing the segment 4. Thus, the working channel section 8 formed by the segment 4 is limited/closed at least partially, preferably completely, on the side of the wedge back section 6 by the back element 7. Optionally, an inner tube 8a is located within the working channel, i.e. running through (all) working channel sections 8 in the longitudinal direction of the endoscope, which seals the working channel, in particular between neighboring segments 4 and neighboring further elements.

A top view of such a segment 4 according to FIG. 2 shows an axial through-hole/through-bore, which forms the working channel section 8 of this segment 4. This through-hole is arranged decentrally offset in the direction of the wedge back section 6 in such a way that the bore/opening diameter extends beyond the outer circumference of the segment 4 and thus breaks up the lateral surface of the segment 4. This creates a kind of crescent shape for the segment 4, with an essentially horseshoe-shaped bulge as the working channel section, the connecting plate 7 only covering a partial circumferential area of the segment 4 in the wedge back section 6 on the outside and—viewed in the circumferential direction-being fixed to the outside of the segment 4 on both sides of the gap-shaped opening. As a result, a radial inner side of the connecting plate 7 forms part of an inner wall of the working channel section 8.

Furthermore, secondary/supply channels 9 are arranged in the endoscope/each segment 4, in particular in the crescent area created by the radial displacement of the through opening, for example for guiding Bowden cables, flushing lines and the like, which have a smaller diameter than the working channel 8. The secondary channels 9 and the working channel 8 are arranged in each segment 4 in such a way that they run in parallel to each other.

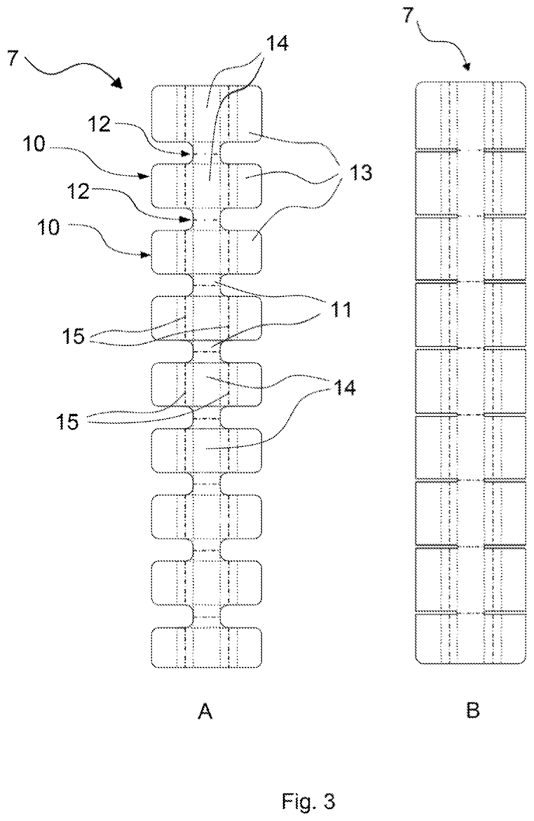

The back element/connecting plate 7, which is described in more detail with reference to FIGS. 3 (left, labeled “A”) and 4, has several axially spaced holding sections 10, on each of which a segment 4 is or will be arranged, as well as hinge sections 11 arranged axially between the holding sections 10, which are arranged correspondingly between two segments 4 and are designed to be bendable/flexible in order to serve as a hinge for folding down the endoscope head 1 by the fold-down mechanism 2. This is illustrated in FIG. 3 by hinge axes 12. Preferably, the back element/connecting plate 7 is tapered/constricted in the area of the hinge sections 11 as viewed in the circumferential direction. Further preferably, the back element/connecting plate 7 is made of an elastic material, for example spring steel, at least in the area of the hinge sections 11.

Furthermore, the/each of the holding sections 10 in particular have two wing-like fastening areas 13 projecting from one another in the circumferential direction, which extend (tab-like) laterally/in the circumferential direction in opposite directions and are designed for fastening the back element 7 to the segments 4 (on both sides of the respective longitudinal slot). A cover region 14 is defined between two fastening regions 13, which is arranged/can be arranged above the aperture/longitudinal slot of the relevant segment 4, which is formed by the working channel 8 in the lateral surface of the corresponding segment 4, as described above. The cover regions 14 and the hinge sections 11 together form a continuous (continuously smooth) radially inner sliding surface 36 of the back element 7, for guiding and sliding medical instruments inserted into the working channel 8, in particular when the fold-down mechanism 2 is folded down (confer FIG. 1, left-hand illustration). Moreover, one of the holding sections 10 is formed at a first (proximal) end of the back element 7 for attachment to the endoscope shaft 20, in particular to the (optional) deflector section 3—if provided—and can be widened for this purpose, for example in the longitudinal and/or circumferential direction of the endoscope relative to the other (segment) holding sections 10.

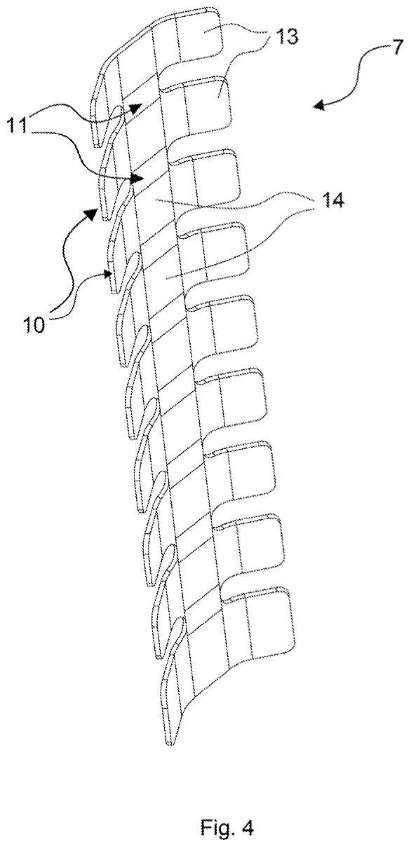

Furthermore, the back element 7 is preferably manufactured from a single plate or sheet, for example punched, as can be seen in FIG. 3, and the holding sections 10 are then bent in order to achieve a curvature of the back element 7, which essentially corresponds to or approximates polygonally an outer circumference of the segments 4. In particular, the fastening regions 13 are bent around a bending axis 15 with respect to the cover regions 14 in order to create the channel-like curvature adapted to the segments 4. A perspective view of the finished back element 7 is shown in FIG. 4.

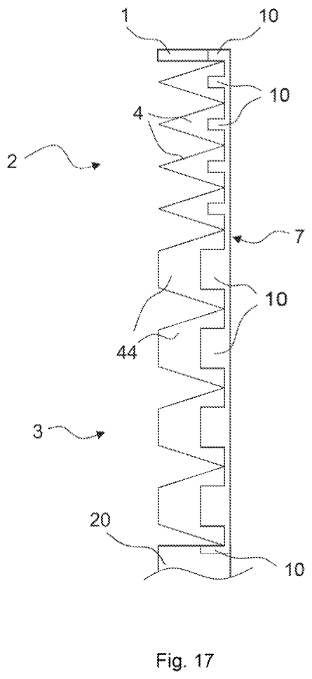

Preferably, the back element 7 can extend along both the fold-down mechanism 2 and the deflector 3 and, as shown in more detail in FIG. 17, connect the endoscope head or the distal head section 1, the segments 4 of the fold-down mechanism 2, the vertebral bodies 44 of the deflector 3 and the (passive) endoscope shaft 20 to each other via corresponding holding sections 10. That is, the back element 7 may have a deflector section (deflector connecting element/back element) that connects the vertebral bodies 44 of the deflector 3. Furthermore, vertebral bodies 44 of the deflector 3, as shown in FIG. 1, can each have a slit or gap-shaped opening in the radial direction at a circumferential position with respect to the endoscope shaft which corresponds to the circumferential position of the wedge back sections 6 of the fold-down mechanism 2 and the back element 7, which extends over the entire length of the respective vertebral body 44 and which preferably connects a working channel extending through the vertebral bodies 44 to an outer side of the vertebral bodies 44. The deflector portion of the back element 7 may cover the slit or gap-shaped openings in the vertebral bodies 44 of the deflector.

A modification of the back element/connecting plate 7 is shown on the right in FIG. 3 (labeled “B”), whereby reference is made to the illustration “A” for reasons of clarity with regard to the reference signs. According to this modification, the tab-shaped fastening regions 13 are wide in comparison to the spaces between the fastening regions 13 or to hinge sections 11. In particular, the spaces are narrow slots or incisions. According to this modification, the fastening regions 13 protrude beyond the segments 4 in the longitudinal direction of the endoscope and overlap each other when the fold-down mechanism 2 is bent.

The function of such a fold-down mechanism 4 can be summarized as follows:

First, as stated above, the endoscope head 1 or the distal endoscope head portion is connected via at least one or more segments 4 to the endoscope shaft 20 or to the optional deflector 3 at the distal end of the endoscope shaft 20. For this purpose, the back element/connecting plate 7 is fixed at its axially spaced holding sections 10 on the endoscope head/distal head section, on the at least one segment 4 and on the endoscope shaft 20 or deflector 3 and thus holds these at least three elements together axially. At the same time, the back element 7 covers the longitudinal gap on the at least one segment 4 and thus forms the sliding surface to the interior of the working channel section 8 partially surrounded by this segment 4.

As soon as an actuating element (pull rope) specific to the fold-down mechanism is manually actuated, the endoscope head/distal head section folds down at the film hinge formed by the back element 7 between the head and the segment 4 arranged immediately proximally thereto, and at the film hinge, which is formed by the back element 7 between the segment 4 and the deflector/endoscope shaft 20 arranged immediately proximally thereto, corresponding to the wedge shape of the at least one segment 4, until the respective end faces of the segments 4 come into contact with an adjacent surface or end face, whereby the working channel 8 formed jointly by the three elements mentioned above is curved.

If, in this fold-down position, a minimally invasive instrument is advanced from the proximal end of the endoscope shaft 20 through the working channel 8 in the distal direction, the tip of the instrument in the area of the three folded-down elements mentioned above inevitably comes into contact with the continuously smooth (stepless) inner side of the back element 7 without it making contact with at least one segment 4 to block the advance. This means that a feed movement can be carried out without any problems even with extremely small fold-down angles in the area of the fold-down mechanism. The decisive factor here is the longitudinal slot-like cut-out of the at least one segment 4, as well as its covering by the back element 7, which simultaneously forms the fold-down hinge or hinges between the elements of the fold-down mechanism.

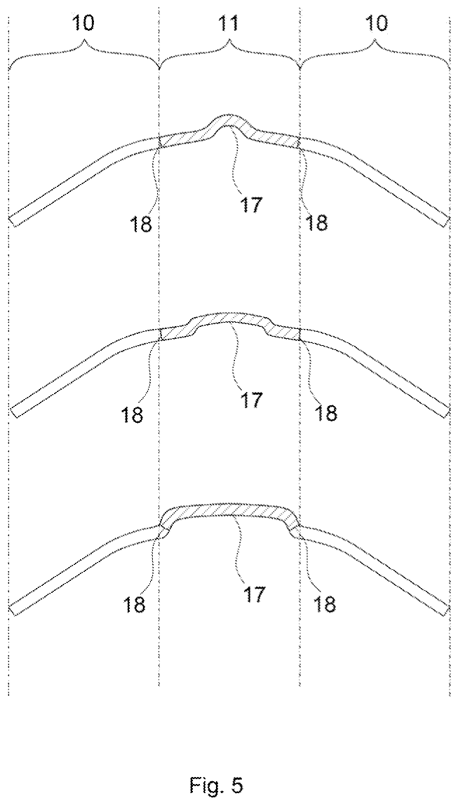

FIG. 5 shows the several optional modifications of the connecting plate/back element 7 in a cross-section through the hinge section 11. The hinge section 11 is curved radially outwards along the circumferential direction of the endoscope/segment and thus forms a (radially inner) groove. The groove has a variable groove radius or radius of curvature in particular. The back element 7 can have identical or differently modified hinge sections along the longitudinal axis of the endoscope. In the latter case, FIG. 5 is to be understood as showing several different hinge sections 11 of a single back element 7.

In the first modification (top of FIG. 5), the groove/curvature radius in the area of a groove center 17 (center of the groove viewed in circumferential direction) is narrow or small (the curvature is large) and increases towards a groove edge 18. The groove radius is therefore large in the area of the groove edge 18 (only relatively slightly curved/provided with a small curvature). In this modification, a restoring force in a stretched position of the fold-down mechanism, exerted by the hinge section 11 in the strongly (in particular maximally) bent/folded-down state of the fold-down mechanism 2 is small, and the restoring force in a slightly (in particular minimally) folded-down state of the fold-down mechanism 2 is large (in relation to the small restoring force in the strongly bent state).

In the second modification (centered in FIG. 5), the groove/curvature radius is large in the area of the groove center 17 and in the area of the groove edge 18 and is narrow or small in an intermediate area between the groove center 17 and the groove edge 18. In this modification, the restoring force in the stretched position exerted by the hinge section 11 in the strongly (in particular maximum) and in the slightly (in particular minimum) bent/folded/folded-down state of the fold-down mechanism 2 is small, and the restoring force in a medium/intermediate folded-down state of the fold-down mechanism 2 is large (in relation to the small restoring force).

In the third modification (bottom of FIG. 5), the groove/curvature radius is large in the area of the groove center 17 and is narrow or small in the area of the groove edge 18. In this modification, the restoring force in the stretched position exerted by the hinge section 11 in the strongly (in particular maximum) bent/folded/folded-down state of the fold-down mechanism 2 is large, and the restoring force in the slightly (in particular minimally) bent/folded/folded-down state of the fold-down mechanism 2 is small (in relation to the large restoring force).

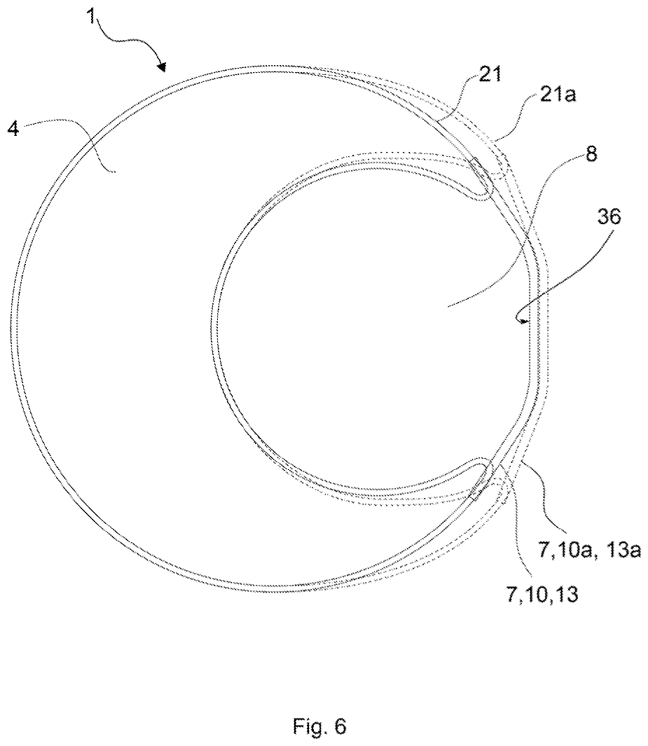

FIG. 6 shows a top view of the endoscope head 1 to illustrate a mode of operation of an optional channel adjustment mechanism of the preferred embodiment. Compared to the illustration in FIG. 2, the endoscope head 1 is simplified for the sake of clarity and only shows the elements relevant for the channel adjustment mechanism. As described above, the segments 4 have a crescent-shaped cross-section, within which the working channel 8 is formed. The ends of the crescent form segment wings 21 which curve towards each other and form side walls of the working channel 8, i.e. which at least partially surround the working channel 8. The connecting plate 7 is attached radially outwardly to the segment wings 21 in each case via its holding section 10, or more precisely, the fastening region 13 thereof. The segment wings 21 can be elastically and/or plastically bent/expanded or bent/contracted radially outwards or inwards in order to increase or decrease the diameter of the working channel 8. In FIG. 6, the widened segment wings 21a are shown as dashed lines.

The back element 7, which curves over (bridges) a gap between the two segment wings 21 to form a side wall of the working channel 8, can also be elastically and/or plastically bent/expanded or bent/contracted radially outwards or inwards. In particular, the holding sections 10 can be bent accordingly. In FIG. 6, the widened holding sections 10a or the widened fastening regions 13a are shown as dashed lines. Thus, the curvature of the back element 7 can be adapted to the segment wings 21 or to the diameter of the working channel 8 in order to be able to safely bridge the gap between them even in case of a narrowed or widened working channel 8 and preferably to correspond to the diameter of the working channel 8.

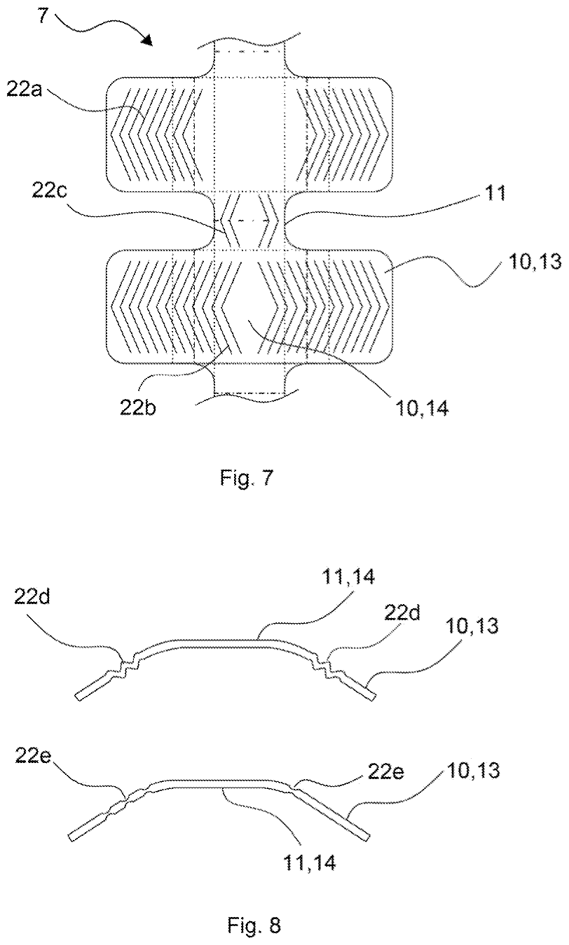

FIG. 7 and FIG. 8 show modifications of a channel adjustment structure 22a to 22d of the back element 7 (connecting plate), which together with the bendable segment wings 21 form the channel adjustment mechanism or are a part thereof. FIG. 7 shows a section of the back element 7 provided with a grid of, for example, V-shaped slots 22a, 22b, 22c, which serves as the channel adjustment structure. The V-shaped slots 22a, 22b, 22c are arranged in the back element 7 in such a way that a tip of the V-shape points outwards (away from the cover regions 14) in the circumferential direction of the segment 4 when the back element 7 is attached thereto. Alternatively, the tip of the V-shape can also point inwards, i.e. towards the cover regions 14 in the circumferential direction of the segment 4. Alternatively, the slits 22a, 22b, 22c may assume any shape which enables/facilitates elastic and/or plastic bending of the back element 7 radially inwards and/or outwards with respect to the endoscope axis.

According to a first modification, channel adjustment structures 22a are provided (exclusively) on the fastening regions 13 of the back element 7, in this example the V-shaped slots, which are shown as an example on the upper holding section 10 in FIG. 7. Alternatively or additionally, according to a second modification, channel adjustment structures 22b (for example the V-shaped slots) are provided on the cover regions 14, which are shown as an example on the lower holding section 10 in FIG. 7. Alternatively or additionally, according to a third modification, channel adjustment structures 22c can also be provided on the hinge sections 11, as shown by way of example in the center in FIG. 7.

Alternatively or additionally, as shown in FIG. 8, a profile cross-section of the back element 7, in particular of the holding sections 10, preferably of the fastening regions 13, can be varied to provide the channel adjustment structure 22d, 22e. The variation of the profile cross-section preferably extends over the entire holding section 10 or the entire fastening region 13 and defines along this extension a bending axis for expanding/contracting the curvature of the holding section 10. For example, the holding section 10 may be corrugated, in particular at a transition of the fastening region 13 to the cover region 14 or at the fastening region 13 near the cover region 14, as shown in FIG. 8 above, to provide a channel adjustment structure 22d according to a fourth modification. Alternatively or additionally, a material thickness (plate thickness/sheet thickness) of the holding section 10, in particular at the transition of the fastening region to the cover region 14 or at the fastening region near the cover region 14, as shown in FIG. 8 below, may be reduced (fold-like) at one or more locations to provide a channel adjustment structure 22e according to a fifth modification. A location with reduced material thickness is formed by a groove running in the longitudinal direction of the endoscope. Preferably, several such grooves run parallel to each other (see FIG. 8 bottom left). However, a single groove 22e on one of the holding sections 10 or fastening regions 13 is also sufficient to provide a channel adjustment structure (see FIG. 8 bottom right). The groove or grooves 22e are arranged here, for example, on a radial inner and outer surface in such a way that a radially outer and a radially inner groove are opposite each other. Alternatively, these can also be arranged offset to one another. Further alternatively, the groove(s) can be provided only radially inside or only radially outside.

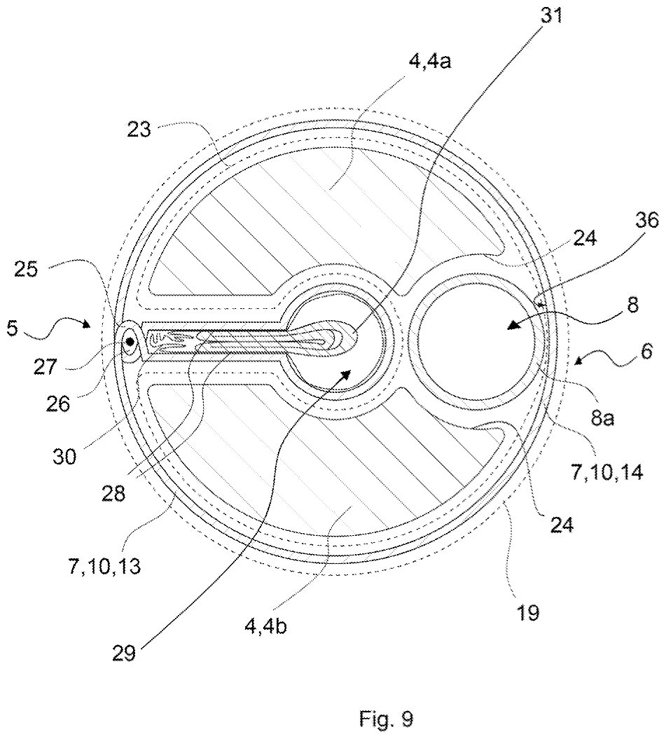

FIG. 9 and FIG. 10 show a cross-section through a fold-down mechanism according to a further embodiment. According to this embodiment, the back element 7 (connecting plate) is formed in the area of the holding sections 10 (i.e., where the segments 4 are attached to the back element 7) as an elastic hollow profile which embraces the segments 4. Each segment is formed by two preferably separate partial segments 4a, 4b, which are each designed to be mirror-symmetrical and are attached (e.g. glued, welded, etc.) to the fastening regions 13 of a corresponding holding section 10. The partial segments are separated from each other (preferably completely) by a gap. Optionally, an enveloping layer 23 is provided at least along the (entire) fold-down mechanism 2 between the segments 4 and the back element 7. The partial segments 4a, 4b form radial recesses 24 in the wedge back section 6, which are arranged opposite each other so as to form the working channel 8 opening at the wedge back section 6. The inner tube 8a runs in the working channel.

The fastening regions 13 of the back element 7 embrace the respective partial segments 4a, 4b and each form an eyelet 25 on the side radially opposite the working channel 8 or on the wedge tip section 5. In a delivery state shown in FIG. 9, the eyelets 25 of the fastening regions 13 lie on top of one another (overlapping one another) when viewed in the longitudinal direction of the endoscope in such a way that the eyelets 25 of the fastening regions 13 of each holding region 10 form a latch channel 26 between them. The fastening regions 13 are designed as spring elements, which are compressed radially inwards in the delivery state and are thus pretensioned. In the delivery state, a latch wire 27 or another flexible, cord-like element is guided through the individual eyelets 25, i.e. through the latch channel 26, and thus holds the fastening regions 13 opposite each other on the wedge tip section 5 together against the pretension of the fastening regions 13.

Optionally, the fastening regions 13 form inner legs 28, which extend substantially in parallel to each other radially inwards through the gap and are connected to each other within the segment 4 or centrally between the partial segments 4a, 4b in order to form a receiving space 29. The sides of the inner legs 28 facing each other and the receiving space 29 are provided with a foil-like (possibly glued-in) lining 30 extending in the longitudinal direction of the endoscope through the (entire) fold-down mechanism 2, which is folded between the inner legs 28 at the wedge tip section 5. Furthermore, an endoscope-longitudinal additional tube 31 is inserted (glued to the inner legs 28) on the sides of the inner legs 28 facing each other or on a corresponding area of the lining 30 and is folded between the inner legs 28 and received in the receiving space 29. Optionally, the additional tube 31 or the lining 30 can be omitted.

If an additional working channel is required in the course of a treatment, for example to be able to insert another instrument or a flush tube, the latch wire 27 is pulled out of the eyelets 25 in a proximal direction, the fastening regions 13a open/spread radially outwards due to their pre-tensioning (Pacman-like) in such a way that the inner legs 28 form an angle between them, as shown in FIG. 10. The folded area of the lining 30 on the wedge tip side is unfolded or stretched between the inner legs 28. Alternatively or additionally, the additional tube 31 unfolds due to the opening of the inner legs 28 and/or its own elasticity and forms an additional working channel 32. The overtube 19 is stretchable to allow the additional working channel 32 to open.

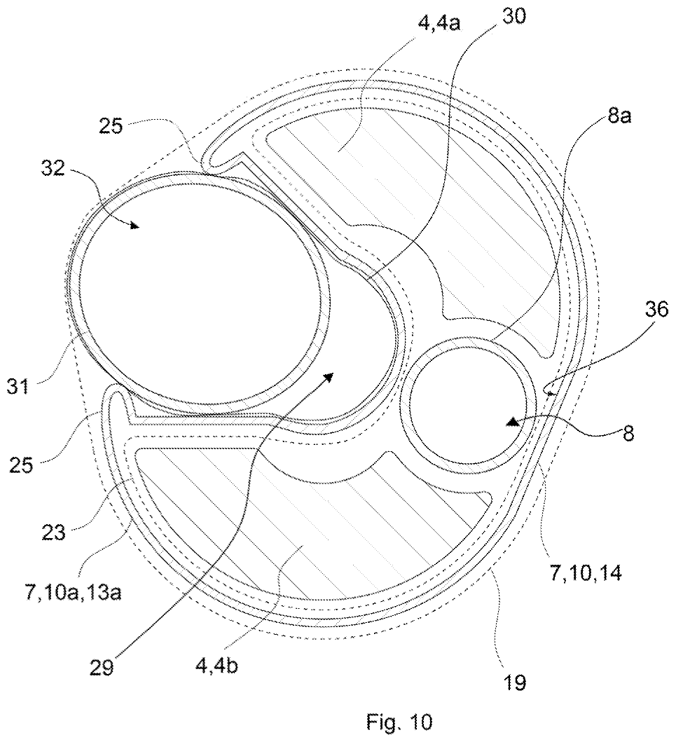

FIG. 11 shows a principle sketch of a fold-down mechanism in a side view according to an alternative embodiment of the disclosure in the extended (top in FIG. 11 or “A”) and folded-down state (bottom in FIG. 11 or “B”). The cylindrical design of the segments 4 in the longitudinal direction of the endoscope and their wedge-shaped design in the side view, as well as their arrangement between a deflector section 3 (or endoscope shaft 20) and an endoscope head (or a distal head section) 1 essentially correspond to the embodiment of the disclosure according to FIGS. 1 to 10, as does the actuation via a corresponding actuating element, which, however, is not shown in FIG. 11. Further elements such as an overtube or the like, which are common to both embodiments, are not shown for reasons of clarity.

In contrast to this, however, the segments 4 are unslotted/unperforated at the wedge back section 6 and are connected to each other at the wedge back section 6 in a pivotable/foldable manner via segment joints 32. This means that the through-openings, which together form the working channel 8, are provided in the respective segments 4 such that the wall of each through opening is closed all the way around. In other words, unlike in the embodiment shown in FIGS. 1 to 10, the through openings do not have a lateral opening or slot. Instead of a back element 7 designed as a connecting plate, a flexible slide-off plate or a flexible slide-off band device 7b is provided which is inserted within the working channel 8 at a position facing the wedge back section 6. At a distal area of the fold-down mechanism 2, preferably at the endoscope head/at the distal head section 1, the slide-off plate/slide-off band device 7b is fixed in position at a fixing point 33, e.g. an bonding, welding or soldering point 33, in/at the working channel 8 (i.e. in/at a through opening). The slide-off plate/slide-off band device 7b extends loosely/longitudinally displaceably through the through openings of the segments 4 or through the working channel 8 proximally from the fixing point 33 and bridges gaps 34 which, as can be seen in the top of FIG. 11 (A), are formed between the respectively adjacent segments 4 in the stretched state of the fold-down mechanism 2. A proximal end 35 of the slide-off plate/slide-off band device 7b lies in the endoscope shaft 20 or deflector section 3 (in each case at its distal end region). Optionally, the slide-off plate/slide-off band device 7b extends through the deflector section 3 and the proximal end 35 is located in the endoscope shaft 20 proximally upstream of the deflector section 3.

If the fold-down mechanism 2 is folded down/bent, as shown in FIG. 11 below (B), the segments 4 come into contact with the respective adjacent segments 4 and the gaps 34 close (partially or completely). The slide-off plate/slide-off band device 7b is held in a fixed position distally at the fixing point 33, whereby the slide-off plate/slide-off band device 7b is bent and essentially follows the curvature of the fold-down mechanism 2 (is pressed into the curvature). Since the slide-off plate/slide-off band device 7b lies loosely in the segments arranged proximally of the endoscope head/distal head section 1 and in the deflector 3/endoscope shaft 20, and due to the rigidity or elasticity of the slide-off plate/slide-off band device 7b, it does not fully abut the segments 4 in the folded-down state, but at most touches the segments 4. As a result, the slide-off plate/slide-off band device 7b does not form the angles and/or gaps 34 that occur at the transition between adjacent segments 4, but instead forms a (uniformly) curved sliding surface 36 on which an instrument pushed through the working channel 8 can slide off evenly.

Furthermore, the fold-down mechanism 2 is shortened by folding down in the area of the slide-off plate/slide-off band device 7b, as the gaps 34 are partially or completely closed. Thus, the proximal end 35 of the slide-off plate/slide-off band device 7b is pushed into the endoscope shaft 20/into the deflector 3 by an insertion distance ΔS when the fold-down mechanism 2 is folded down. The insertion distance ΔS essentially corresponds to the summed width of the gaps 34 between the segments 4 in the area of the working channel walls facing the wedge back section 6 in the stretched state of the fold-down mechanism 2, and a shortening of the section of the slide-off plate/slide-off band device 7b running through the segments 4 due to the curvature of the slide-off plate/slide-off band device 7b in relation to the straight (in the longitudinal direction of the endoscope) working channel walls of the segments 4.

If the fold-down mechanism 2 is stretched again, the slide-off plate/slide-off band device 7b stretches, in particular due to its elasticity/restoring force.

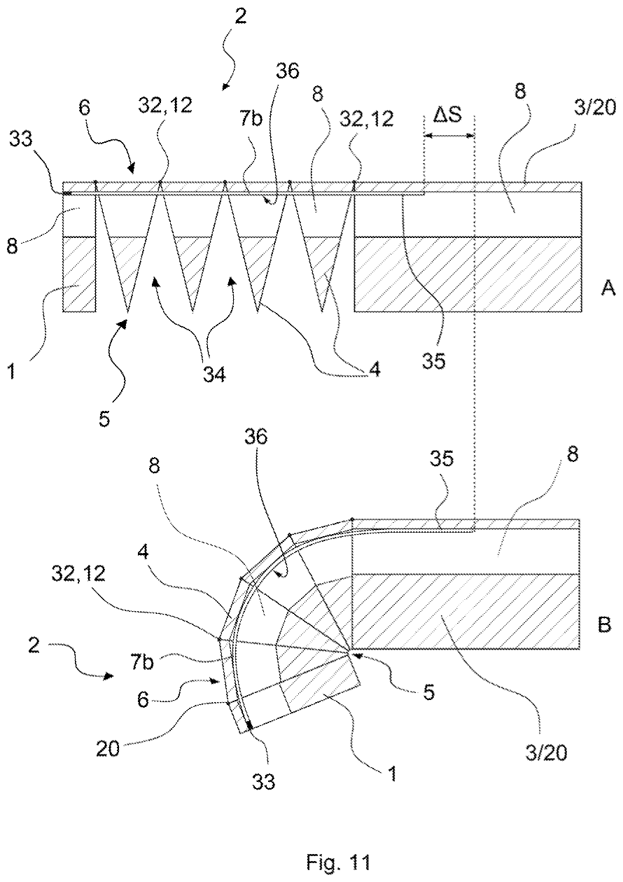

FIG. 12 shows a preferred embodiment of the slide-off band device 7b (left) from FIG. 11 and its position in the cross-section of the fold-down mechanism 2 (right) between an inner tube 8a of the working channel 8 and an inner wall of the working channel 8 on the wedge back side 6 of the segments 4. The slide-off band device 7b is fork-shaped and has several parallel longitudinal strips as longitudinal elements 37, which are connected to each other at their distal end via a transverse web 38. At a fixing point 33 of the transverse web 38, the slide-off band device 7b is connected to the distal region of the fold-down mechanism 2. On a side facing the working channel 8, the longitudinal elements 37 form sliding surfaces 36 for an instrument pushed through the working channel 8.

FIG. 13 shows a further preferred embodiment of the slide-off band device 7b (left) from FIG. 11 and its position in the cross-section of the fold-down mechanism 2 (right) between an inner tube 8a of the working channel 8 and an inner wall of the working channel 8 on the wedge back side 6 of the segments 4. The slide-off band device 7b is formed by a plurality of parallel wires 37 as longitudinal elements, each of which forms a fixing point 33 at its distal end, via which the slide-off band device 7b is connected to the distal region of the fold-down mechanism 2. In particular, the wires 37 are at least partially guided in grooves in the inner wall of the working channel 8. The wires 37 may optionally be provided in addition to the rectangular/ribbon-shaped or fork-shaped slide-off band device (arranged here in schematic representation between two groups of the wires 37). On a side facing the working channel 8, the wires 37 form sliding surfaces or sliding lines 36 for an instrument pushed through the working channel 8.

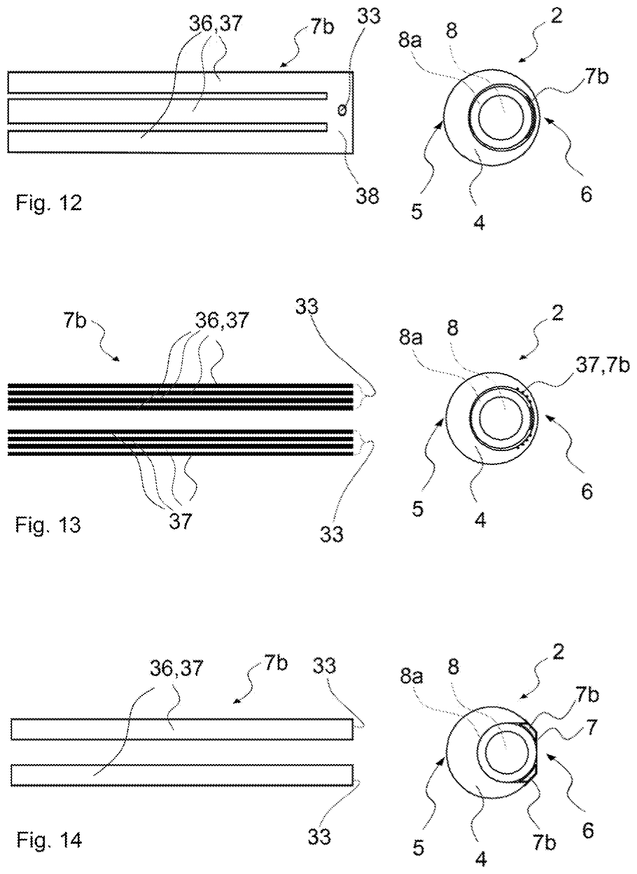

FIG. 14 shows a further preferred embodiment of the fold-down mechanism 2 of the present disclosure, which essentially corresponds to the embodiment according to FIG. 2, with differences explained below. A width of the slot on the wedge back section 6 of the segments 4 corresponds to a diameter of the working channel 8 (see cross-sectional view on the right). A space is formed which is bounded radially on the inside by an inner tube 8a of the working channel 8, radially on the outside by the connecting plate/back element 7 and in the circumferential direction on a side facing away from the wedge back section 6 by the segment 4. A slide-off band device 7b is provided in this space in such a way that it extends along the fold-down mechanism 2 (similar to the slide-off band device 7b in FIG. 11). On the left, the slide-off band device 7b is depicted in a top view. It forms two parallel longitudinal elements 37, which have distal fixing points 33, via which the longitudinal elements 37 or the slide-off band device 7b is connected to the distal area of the fold-down mechanism 2. On a side facing the working channel 8, the longitudinal elements 37 form sliding surfaces 36 for an instrument pushed through the working channel 8.

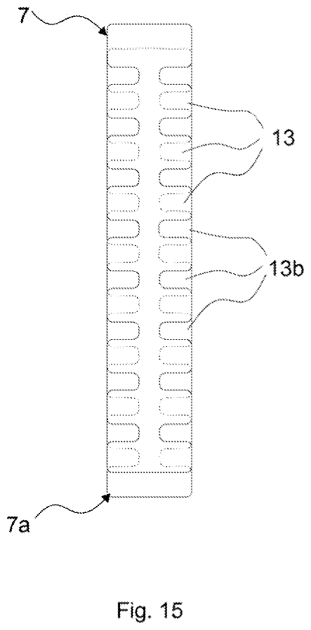

FIG. 15 shows an aspect of a modification of the embodiments according to FIG. 1 to FIG. 8, in particular according to FIG. 1. More precisely, according to this aspect, the back element/connecting plate 7 according to FIG. 3 (left, A) is provided as a first back element/first connecting plate 7. A second back element/a second connecting plate 7a (shown with dashed lines where it is covered by the first connecting plate 7) is provided, which is arranged offset in the longitudinal direction of the endoscope relative to the first connecting plate 7 in such a way that the tabs 13b of the second connecting plate 7a overlap spaces between the tabs or fastening sections 13 of the first connecting plate 7. The segments 4 are preferably attached only to the tabs or fastening sections 13 of the first radially outer connecting plate 7 in the assembled state, or alternatively are preferably attached alternately to the tabs or fastening sections 13 of the first connecting plate 7 and to the tabs 13b of the second connecting plate 7a.

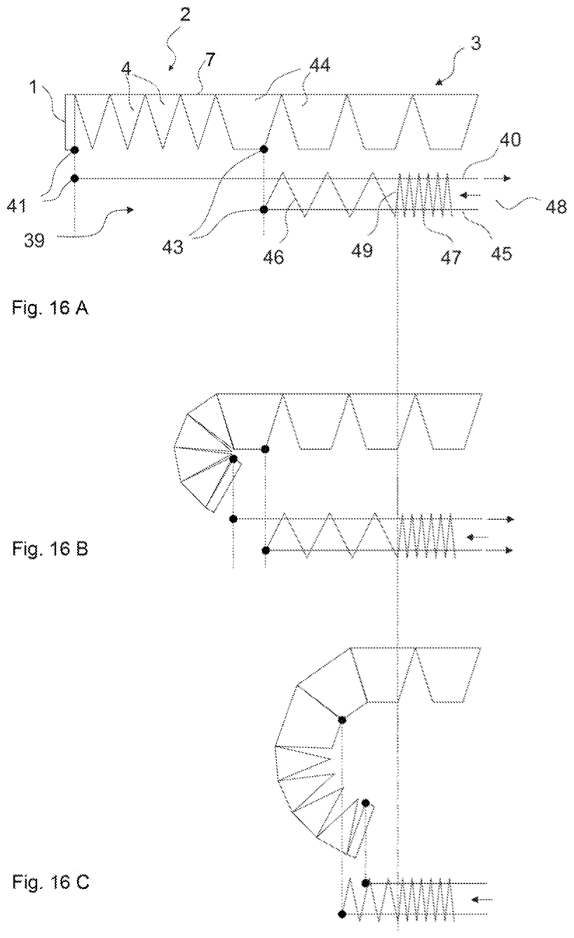

FIG. 16A to FIG. 16C schematically show an actuating sequence of an actuating mechanism for actuating an endoscope according to one of the embodiments of FIGS. 1 to 15, reference signs only being shown in FIG. 16A for reasons of clarity. For reasons of clarity, the fold-down mechanism 2 is only shown schematically in this illustration and moreover has the connecting plate 7 and/or the slide-off band device 7b according to one of the other embodiments described. In the case of the connecting plate 7, this essentially follows the straight wedge back sections 6 of the segments 4 or vertebral bodies 44 and is only bent at the hinge sections in between.

In FIG. 16A, the endoscope according to one of the above embodiments is shown in a neutral/unactuated (stretched) position. An actuating mechanism 39 for actuating/deflecting the fold-down mechanism 2 and the deflector 3 is shown in parallel to the endoscope. The actuating mechanism 39 has a control wire 40, the distal end of which is connected (directly attached) to the distal head section/endoscope head 1 at a first reference point 41.

Furthermore, the actuating mechanism 39 has an actuating spring 42 the distal end of which is connected (directly attached) to a distal-most vertebral body 44 of the deflector 3 at a second reference point 43. A distal end of a locking wire 45 of the actuating mechanism 39 is connected to (directly attached to) the distal end of the actuating spring 42. The actuating spring 42 has a distal spring portion 46 with a large pitch/coil spacing and a proximal spring portion 47 with a small pitch/coil spacing. In particular, a coil spacing of the proximal spring portion 47 is zero, i.e. the coils of the actuating spring 42 are in contact with one another. The locking wire 45 locks/limits an expansion of the actuating spring 42, in particular of the distal spring area 46, and holds it/they in a compressed position. A support spring/compression spring 48 (shown symbolically in FIGS. 16A to C by a force arrow pointing in the distal direction) supports the actuating spring 42 from the proximal direction.

If, starting from the neutral/unactuated position, the control wire 40 is pulled/actuated in the proximal direction (shown in FIG. 16A by an arrow pointing proximally on the control wire 40), the fold-down mechanism 2 is folded/bent, as shown in FIG. 16 B. A pre-tensioning force, which acts on the most distally arranged vertebral body 44 of the deflector 3 by the locking wire 45 and/or by the support spring/compression spring 48, prevents the deflector 3 from bending due to the actuation of the control wire 40.

If, as shown in FIG. 16 B by an arrow on the blocking wire 45 pointing in the proximal direction, the blocking wire 45 is then pulled in the proximal direction within a first actuation range (if it is actuated in the proximal direction), the distal spring region 46 is first compressed further, as shown in FIG. 16 C, and the vertebral bodies 44 of the deflector 3 arranged furthest distally are thus bent. In particular, the vertebral bodies 44 of the deflector 3 are bent/which are located distally from a transition region 49 between the proximal spring region 47 and the distal fault region 46. In the present example, the control wire 40 is pulled in the proximal direction (shown in FIG. 16 B by an arrow on the control wire 40 pointing in the proximal direction) at the same time as the locking wire 45 is actuated in the first actuation region in order to compensate for a change in length/length displacement of the control wire 40 due to the unwinding of the deflector 3, and to maintain the bending of the fold-down mechanism 2.

Alternatively, if the control wire 40 is not pulled or is pulled less in the proximal direction during actuation of the locking wire 45 in the first actuation area, the curvature of the fold-down mechanism 2 opens completely or partially. For example, a curvature of the fold-down mechanism 2 in connection with the distal vertebral bodies 44 of the deflector 3 of 180° can be obtained in this way.

If, based on FIG. 16 C, the locking wire 45 is then pulled/actuated further in the proximal direction, the coils of the distal spring region 46 come into contact. This limits the first actuation range of the locking wire 45. If the locking wire of 45/the actuation spring 42 is then pulled/actuated further in the proximal direction (second actuation range), the vertebral bodies 44 of the deflector 3 arranged furthest proximally are also bent (not shown here).

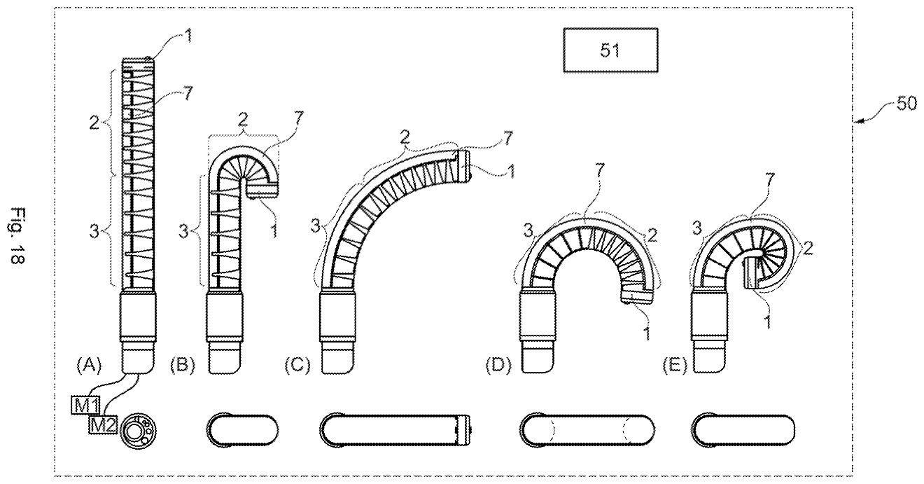

FIG. 18 shows an endoscope with a distal endoscope head or head section 1, a fold-down mechanism 2 arranged proximally thereto and a deflector section (in short: deflector) 3 arranged proximally thereto in various adjustable modes or operating states. A side view of the endoscope and a top view of the endoscope from a direction, in which the endoscope extends in the stretched state, is shown of each mode or operating state. In particular, it is an endoscope according to one of the preceding figures.

In a neutral mode, which is shown on the far left and labeled (A), the endoscope can be controlled in such a way that the folding mechanism 2 and the deflector 3 are stretched, as shown here. Optionally, they can even be slightly overstretched, i.e. bent slightly backwards in the direction of any back element 7 provided (see description above).

In a second mode, characterized by (B), the endoscope is controllable in such a way that only the fold-down mechanism 2 is bent, while the deflector 3 remains stretched or unactuated. As can be seen in the associated top view, the lateral extension of the endoscope in this mode is relatively small.

In a third mode, characterized here by (C) and (D), the endoscope is controllable in such a way that the fold-down mechanism 2 and the deflector 3 can be bent synchronously with each other, i.e. essentially simultaneously with a constant/equal/continuous degree of curvature. In the associated top views, it is to be recognized that a lateral extension of the endoscope in this mode is relatively large. In this way, the endoscope can, for example, achieve a 90° curve in its distal area (see view (C)) or a 180° curve (see view (D)). In the latter case, for example, the deflector 3 can be maximally bent.

In a fourth mode, which is shown on the far right and labeled by (F), the deflector 3 is, for example, fully bent by a control in the third mode, and the endoscope can then be controlled in such a way that the fold-down mechanism 2 can be further deflected so that the position shown here can be achieved in which both the deflector 3 and the fold-down mechanism 2 are bent to the maximum.

The endoscope can be part of a treatment system 50, which further comprises a control unit/control device 51, which is configured to control the endoscope selectively in one of the modes described above.

The endoscope also has at least one drive M1 for driving the deflector 3 and a drive M2 for driving the fold-down mechanism 2, which are only shown schematically here. The control device 51 is configured to control the drives M1 and M2 at different speeds and independently of each other depending on the mode.

### LIST OF REFERENCE SIGNS

1 Endoscope head/distal head section
2 Fold-down mechanism
3 Deflector section
4 Segments
4a, 4b Partial segments
5 Wedge tip section
6 Wedge back section
7 Connecting plate/back element
7a Second connecting plate/back element
7b Flexible slide-off plate/slide-off band device
8 Working channel
8a Inner tube
9 Secondary channels
10 Holding section
10a Expanded holding section
11 Hinge section
12 Hinge axis
13 Fastening region/tabs
13a Expanded fastening region
13b Tabs of the second back element/connecting plate
14 Cover region
15 Bending axis
16 Actuating element
17 Groove center
18 Groove edge
19 Endoscope shell/overtube
20 (Passive) endoscope shaft
21 Segment wing
21a Widened segment wing
22a-e Channel adjustment structures
23 Shell layer
24 Recess
25 Eyelet
26 Latch channel
27 Latch wire
28 Inner leg
29 Receiving space
30 Lining
31 Additional tube
32 Segment joints
33 Bonding, welding or soldering point/fixing point
34 Gaps between segments
35 Proximal end/proximal end region of the back element
36 Sliding surface
37 Longitudinal strip
38 Transverse web
39 Actuating mechanism
40 Control wire
41 First reference point
42 Actuating spring
43 Second reference point
44 Vertebral body
45 Locking wire
46 Distal spring region
47 Proximal spring region
48 Support spring/compression spring
49 Transition region
50 Treatment system
51 Control device
ΔS Insertion distance

1.-21. (canceled)

22. An endoscope including:
an endoscope shaft comprising a distal manually actuable fold-down mechanism for the controlled foldable or angularly adjustable holding of an endoscope head or distal head section on an endoscope section proximally upstream of the fold-down mechanism, the fold-down mechanism having a number of axially successive substantially cylindrical segments which are actively and angularly adjustable relative to one another via at least one common actuating element,
wherein the segments each have:
two axial end faces aligned substantially wedge-shaped with respect to one another,
a cylinder jacket having a wedge back section at a section of the cylinder jacket having a maximum axial length, and
at least one through opening in a longitudinal direction of the endoscope such that the segments jointly form a working channel, wherein the through opening in the wedge back section of each segment breaks through the cylinder jacket in a radial direction of the endoscope, thereby forming a longitudinal slot; and
wherein the wedge back sections of each segment are connected to one another in the longitudinal direction of the endoscope by a flexible or elastic connecting plate, which is fixed to an outer side of each segment and covers the respective longitudinal slots, wherein the connecting plate forms a sliding surface along which medical instruments can be inserted, wherein the sliding surface runs continuously in the working channel, wherein the connecting plate also forms hinge sections, which are located between adjacent segments, that are connected to one another for folding down two respective adjacent segments.

23. The endoscope according to claim 22, wherein the connecting plate at least partially covers the working channel in the wedge back section.

24. The endoscope according to claim 22, wherein the connecting plate includes tab-shaped holding sections for attachment to the segment and to the distal end section of the endoscope section.

25. The endoscope according to claim 22, wherein the connecting plate is resilient at least at the hinge sections.

26. The endoscope according to claim 25, wherein the hinge sections serve as a return element which is configured to return the fold-down mechanism from a folded-down position to a substantially forwardly aligned or stretched position.

27. The endoscope according to claim 25 wherein the hinge sections are curved or bent radially outwards in a circumferential direction, such that each hinge section forms a groove extending in the longitudinal direction of the endoscope with a specific, constant or variable, groove radius.

28. The endoscope according to claim 22, wherein the connecting plate is tapered at the hinge sections in a circumferential direction.

29. The endoscope according to claim 22, wherein the connecting plate forms a smooth central section, which is continuous in the longitudinal direction of the endoscope and forms the sliding surface, from which tab-shaped fastening regions protrude symmetrically on both sides, which tab-shaped fastening regions serve to fasten the segments and are bent towards each other with respect to the central section.

30. The endoscope according to claim 29, wherein the connecting plate is a first, radially outer connecting plate, and wherein the fold-down mechanism further comprises a second, radially inner connecting plate, which has a second central section extending in the longitudinal direction of the endoscope and tabs projecting laterally therefrom, and the second, radially inner connecting plate is arranged offset in the longitudinal direction of the endoscope relative to the first, radially outer connecting plate, such that the tabs of the second, radially inner connecting plate cover intermediate spaces between the fastening regions of the first, radially outer connecting plate.

31. The endoscope according to claim 22, wherein the fold-down mechanism is configured to hold the endoscope head at the distal end of an endoscope deflector, which is interposed between the fold-down mechanism and the endoscope shaft and is bendable by way of a separate actuating element.

32. The endoscope according to claim 31, wherein when the fold-down mechanism is completely folded down, a bending radius of the fold-down mechanism, is smaller than a bending radius of the endoscope deflector when the endoscope deflector is completely bent down.

33. The endoscope according to claim 22, further comprising a channel adjustment mechanism configured to increase or decrease a diameter of the working channel, such that the wedge back section of the segments and the connecting plate are configured to be bendable in an open position, a closed position, a radially inward direction, a radially outward direction, or a combination thereof with respect to the longitudinal direction of the endoscope.

34. The endoscope according to claim 33, wherein the connecting plate comprises channel adjustment structures for locally reducing the bending stiffness of the connecting plate such that the connecting plate is configured to be defined in a bendable open position, a bendable closed position, in a radially inward direction, a radially outward direction, or a combination thereof with respect to the longitudinal direction of the endoscope.

35. The endoscope according to claim 22, wherein each segment comprises two partial segments connected to each other via the connecting plate, wherein the connecting plate is configured as an expansion spring and is configured to selectively expand a gap between the partial segments, such that a space for an additional working channel is provided therebetween.

36. The endoscope according to claim 22, further comprising a flexible slide-off band device that extends in the longitudinal direction of the endoscope along several of the segments and extends along the working channel, such that the slide-off band device at least partially covers the connecting plate, the wedge back section of the respective segments, or a combination thereof.

37. An endoscope comprising:
an endoscope shaft having a distal manually actuable fold-down mechanism for the controlled foldable or angularly adjustable mounting of an endoscope head or distal head section on an endoscope section that is proximally upstream of the fold-down mechanism, wherein the fold-down mechanism has a number of axially successive and substantially cylindrical segments which are actively angularly adjustable relative to one another by way of at least one common actuating element, wherein the segments are hinged to one another, and wherein each segment has two axial end faces aligned substantially in a wedge shape relative to one another and a cylinder jacket that defines a wedge back section at a section of the cylinder jacket with maximum axial length, wherein each segment has at least one through opening in a longitudinal direction of the endoscope in such a way that the segments jointly form a working channel with a working channel wall; and
a flexible slide-off band device extending in the longitudinal direction of the endoscope along several segments and in the working channel, such that the slide-off band device covers a wedge-back section of the working channel wall, thereby forming a continuous, smooth sliding surface along which medical instruments can be inserted into the working channel.

38. The endoscope according to claim 37, wherein the slide-off band device is fixed to a distal end region of the fold-down mechanism or to one of the segments arranged furthest distally relative to the slide-off band device, and the slide-off band device lies displaceably in the longitudinal direction of the endoscope in the through-openings of a number of segments arranged further proximally, such that the slide-off band device can slip in the working channel when the fold-down mechanism is bent to avoid stretching or compression of the slide-off band device in the longitudinal direction of the endoscope.

39. The endoscope according to claim 37, wherein a proximal end of the slide-off band device protrudes into the endoscope section proximally upstream of the fold-down mechanism so as to be displaceable in the longitudinal direction of the endoscope.

40. The endoscope according to claim 37, wherein the slide-off band device is elastic and extends substantially straight in a rest position, so that the slide-off band device is driven into the unbent rest position when the fold-down mechanism is reset from a folded-down position into a substantially forwardly aligned or stretched position.

41. The endoscope according to claim 37, wherein the slide-off band device is substantially inextensible in the longitudinal direction of the endoscope.

42. The endoscope according to claim 37, wherein the slide-off band device comprises a plurality of longitudinal elements which extend in the longitudinal direction of the endoscope.
